(19) United States
(12) Patent Application Publication
(10) Pub. No.: US 2009/0208919 A1
(43) Pub. Date: Aug. 20, 2009

(54) PARTICLE MATRIX FOR STORAGE OF BIOMOLECULES

(75) Inventors: Joseph G. Utermohlen, Tucson, AZ (US); Michael E. Hogan, Tucson, AZ (US)

Correspondence Address: ROSENBAUM & ASSOCIATES, P.C., 650 DUNDEE ROAD, SUITE #380, NORTHBROOK, IL, 60062, US

(73) Assignee: Argylla Technologies, LLP, Tucson, AZ (US)

(21) Appl. No.: 12/015,402

(22) Filed: Jan. 16, 2008

Related U.S. Application Data
(63) Continuation-in-part of application No. 11/338,124, filed on Jan. 23, 2006.
(60) Provisional application No. 60/885,206, filed on Jan. 16, 2007.
(60) Provisional application No. 60/646,155, filed on Jan. 21, 2005.
(60) Provisional application No. 60/701,630, filed on Jul. 22, 2005.

(51) Int. Cl. A01N 1/02; C12N 5/06; C09K 3/00

(52) U.S. Cl. 435/2; 435/403; 252/182.11; 252/182.35; 252/182.32; 252/182.33; 252/182.3; 252/182.12; 977/811

(57) ABSTRACT

Matrices for manipulation of biopolymers, including the separation, purification, immobilization and archival storage of biopolymers is disclosed.

### CROSS-REFERENCE TO RELATED APPLICATIONS

This application claims priority to U.S. Provisional Patent Application Ser. No. 60/885,206, filed on Jan. 16, 2007. This application also claims priority, as a continuation-in-part, to U.S. patent application Ser. No. 11/338,124, filed on Jan. 23, 2006, and published as U.S. Patent Application Publication US 2006/0177855 A1 on Aug. 10, 2006. U.S. patent application Ser. No. 11/338,124 relates to and claims priority from Provisional Patent Application Ser. No. 60/646,155, filed Jan. 21, 2005 and Provisional Patent Application Ser. No. 60/701,630, filed Jul. 22, 2005. Each of these applications is incorporated herein by reference in its entirety.

### FIELD OF THE INVENTION

Matrices for the manipulation of biopolymers, including the separation, purification, immobilization, and archival storage of biopolymers are provided.

### BACKGROUND

DNA, RNA, immunoglobulins, and proteins are classes of polymeric biomolecules (“biopolymers”) of particular importance in modern biochemical and molecular biological methods and processes. Specifically, biopolymers play critical roles in various subcellular processes including the preservation and transmission of genetic information, the production of proteins, and the formation of enzymes.

Due to the importance of these biopolymers in various biological processes, a wide variety of techniques have been developed to physically bind these classes of molecules in order to manipulate them for immobilization, purification, concentration, archival storage, etc. Biopolymer immobilization, separation, concentration, purification, and storage are employed across a wide range of commercial applications, including, for example, forensics, pharmaceutical research and development, medical diagnostics and therapeutics, environmental analysis, such as water purification or water quality monitoring, nucleic acid purification, proteomics, and field collection of biological samples. Thus, a need exists for efficient, simplified processing of clinical, environmental and forensic samples, especially for samples containing only nanogram amounts of nucleic acid or protein.

Many of the conventional techniques for manipulating and storing biopolymers are costly, complex, and are of limited efficiency, particularly when handling small quantities of biopolymers. In light of the importance of biopolymers to modern biological research such as the development of new therapeutic treatments, drugs, etc., there is a need for alternate methods for manipulating such biopolymers that address these various deficiencies in current techniques.

### SUMMARY

The present invention provides compositions, devices and methods useful for storing biomolecules. More specifically, nanoparticles according to the present invention are used for the stabilization, storage, and retrieval of biomolecules, including nucleic acids and proteins.

In one embodiment, ceramic particles are provided for biomolecule storage. The state of the ceramic particles is reversible, as they may exist in a dry state or in suspension in solution.

In one embodiment, particles for biomolecule storage of a nanoparticle scale are provided. In another embodiment, particles for biomolecule storage of a microparticle scale are provided. In another embodiment, particles of a larger scale than nano- or micro-particles for biomolecule storage are provided.

In one embodiment, passivated nanoparticles are provided for biomolecule storage. Passivation of the nanoparticles yields the nanoparticles substantially inert to the biomolecules.

In another embodiment, methods of storing biomolecules and retrieving the stored biomolecules are provided. Methods of storing biomolecules in a dried state are provided.

In another aspect, the present invention provides ceramic particles, and methods for making and using such particles, that are specifically optimized for the manipulation and storage of specific types of biomolecules. Various preferred embodiments are specifically directed to the storage of DNA, RNA, and proteins in a dry state.

Another aspect of the present invention is directed to the surface modification of nanoparticles using an adaptation of passivation chemistry that relies on oxyanions and other anions to modify the particle surface for biochemical manipulations.

Another aspect of the present invention is directed to using the nanoparticles as a solid phase platform for the stabilization of proteins and nucleic acids for storage applications.

Another aspect of the present invention is directed to using the storage particles as a solid phase platform for purifying or detecting specific biomolecules via electrophoresis of the storage particles.

### BRIEF DESCRIPTION OF FIGURES

FIG. 1 is a schematic view of a nanoparticle storage matrix.

FIG. 2 is a schematic view of DNA storage clefts created from interstices among nanoparticles.

FIG. 3 is a gel of PCR products from DNA recovered from dry state storage for 1 day and 3 days using a nanoparticle matrix composed of tungsten oxide passivated with borax. (See Example 17.)

FIG. 4 is a gel of PCR products from DNA recovered from dry state storage for 1 day and 3 days using a nanoparticle matrix composed of zirconium oxide passivated with borax. (See Example 18.)

FIG. 5 is a gel of PCR products from DNA recovered from dry-state storage for 10 and 34 days using nanoparticles composed of zirconium oxide or nanoparticles composed of tungsten oxide. (See Example 19.)

FIG. 6 is a gel of PCR products from DNA recovered after 7 days of dry storage using nanoparticles composed of zirconium oxide passivated with borax, with the addition of glycerol as a plasticizer. (See Example 20.)

FIG. 7 is a gel of PCR products from DNA recovered after 10 days of dry state storage using a nanoparticle matrix composed of zirconium oxide passivated with borax. (See Example 21.)

FIG. 8 is a gel of PCR products from DNA recovered after 12 days of dry state storage using a nanoparticle matrix composed of zirconium oxide passivated with borax. (See Example 22.)

FIG. 9 is a gel of PCR products from DNA recovered after 25 days of dry state storage using a nanoparticle matrix composed of zirconium oxide passivated with borax. (See Example 23.)

FIG. 10 is a gel of PCR products from DNA recovered after 52 days of dry state storage using a nanoparticle matrix composed of zirconium oxide passivated with borax. (See Example 24.)

FIG. 11 is a gel of PCR products from DNA recovered after 72 days of dry state storage using a nanoparticle matrix composed of zirconium oxide passivated with borax. (See Example 25.)

FIG. 12 is a gel of PCR products from DNA recovered after 72 days of dry state storage using a nanoparticle matrix composed of zirconium oxide passivated with borax. (See Example 26.)

FIG. 13 is a gel of PCR products from DNA recovered after 112 and 118 days of dry state storage using a nanoparticle matrix composed of zirconium oxide passivated with borax. (See Example 27.)

FIG. 14 is a gel of PCR products from DNA recovered after 100 days of dry state storage using a kaolin particle matrix. (See Example 28.)

FIG. 15 is a gel of PCR products from Buccal DNA recovered after 10 days of dry state storage using a kaolin particle matrix. (See Example 30.)

FIG. 16 is a gel of PCR products from Blood Lysate DNA recovered after 1 day of dry state storage using a kaolin particle matrix. (See Example 31.)

FIG. 17 is a gel of PCR products from Blood Lysate DNA recovered after 10 days of dry state storage using a kaolin particle matrix. (See Example 32.)

FIG. 18 is a gel of PCR products from Whole Blood DNA recovered after 36 days of dry state storage using a kaolin particle matrix. (See Example 33.)

FIG. 19 is a gel of PCR products from Buccal DNA recovered after dry state storage using a kaolin particle matrix. (See Example 34.)

FIG. 20 is a gel of PCR products from RNA recovered after dry state storage using a nanoparticle matrix composed of zirconium oxide passivated with borax. (See Example 35.)

### DETAILED DESCRIPTION OF THE PREFERRED EMBODIMENTS

The present invention provides particles matrices, and methods for making and using particle matrices, that are specifically optimized for the manipulation and storage of specific types of biomolecules. The optimization parameters include, for example, the composition and size of the particles, the nature of the particle coating, and the type of associated small-molecule solute in the storage complex. Optimization of these parameters for various applications can result in extending the storage lifetime of biomolecules, increasing the range of temperatures at which the biomolecules can be stored, and preserving the condition of the biomolecules during storage. Various preferred embodiments are specifically directed to the storage of DNA, RNA, and proteins in a dry state.

In one embodiment, ceramic particles of a nanoparticle scale are used. The nanoparticles are approximately spherical in shape, ceramic in composition, and have a diameter from about 20 nm to about 1000 nm. The ceramic can be made of metal oxide or aluminum silicates, such as tungsten oxide, zirconium oxide, or kaolin. Preferably, the surface of these ceramic particles is passivated, or stably treated, with an oxyanion (such as borate, phosphate, sulfate, and citrate) to weaken or eliminate biomolecule interaction with the nanoparticle surface. Such passivated nanoparticles are mixed as a colloidal suspension with the biomolecule to be stored, then allowed to air dry to form a fluid-free, air-dried storage matrix comprised of the nanoparticle and the biomolecule. The drying process results in a dried solid matrix where the biomolecule is encapsulated within the interstitial spaces formed between closely packed nanoparticles. Upon re-hydration, the nanoparticle storage matrix is disrupted by wetting, and the nanoparticles are dispersed as a colloidal suspension. In that re-hydrated suspension, the biomolecule may be harvested away from the matrix components by centrifugation of the nanoparticles, thereby forming a pellet substantially free from the biomolecule, and leaving the biomolecule in solution.

Porous substances that are currently used for dry state biomolecule storage, such as paper or sponge, form an irreversible, porous storage matrix. That is, once hydrated, these porous matrices remain intact under all ordinary conditions of biomolecule sample handling. Thus, biomolecules stored in such an irreversibly porous matrix can become trapped within the pores and may, consequently, diffuse slowly or incompletely from the porous matrix back into the fluid phase upon rehydration. In contrast, non-porous nanoparticles can spontaneously assemble during the process of air-drying to form a matrix which approximates a 3-dimensional volume of closely packed spheres with spaces formed between the spheres available to sequester biomolecules. Upon re-hydration, that matrix of closely packed spheres dissociates to form a dilute aqueous suspension, thereby allowing the stored biomolecules to partition freely into to the fluid phase, free from diffusional impediment imposed by the nanoparticles.

One aspect of the present invention provides the use of spherical nanoparticles composed of branched polymers of sugars, such as a polysucrose polymer described as Ficoll, as the nanoparticle matrix. Polysucrose has been used for cryogenic stabilization of live cells; these same properties of polysucrose are also useful for the dry state storage of biomolecules. A dry state matrix comprising Ficoll spheres would be particularly advantageous for the storage and retrieval of a number of biomolecules including proteins. Upon re-hydration, the matrix of closely packed spheres dissociates to form a dilute aqueous suspension, allowing the stored biomolecules to partition freely into to the fluid phase, free from diffusional impediment imposed by the nanoparticles. The interaction and surface reactivity of polysucrose with other drying buffer additives, such as borate, can further enhance the impermeability of the nanoparticle matrix to outside elements.

Additionally, larger more porous beads are useful in making microparticle matrices. One such particle is the porous spherical structure associated with Sephadex chromatography beads. The utility of such a matrix is that it can easily form a particle based matrix that is readily disrupted into discrete units once it is rehydrated. One advantage of a porous matrix, such as Sephadex, is that the pores can be used to exclude those biomolecules that are to be retrieved upon rehydration, whereas smaller contaminating components can be partially partitioned with the porous matrix and be eliminated from the desired larger biomolecule.

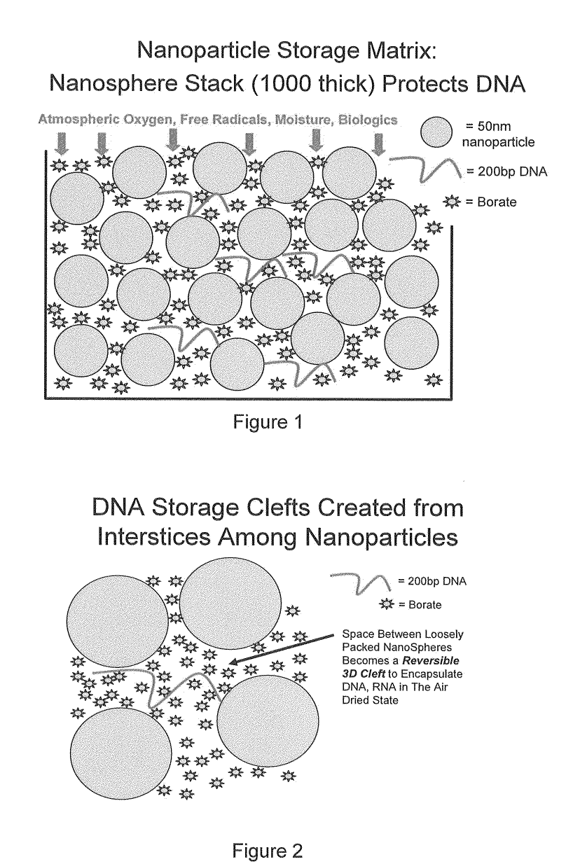

When spherical or nearly spherical non-porous particles are allowed to form a closely packed phase, a fraction of the total phase volume remains unoccupied by particles. For the orderly face-centered packing of spheres (like oranges in a grocery shelf), Gauss first calculated the unoccupied volume to be 26% (Conway and Sloane, 1993, Sphere Packings, Lattices, and Groups, 2nd ed. New York: Springer-Verlag). More recently, Torquato at Princeton has calculated the unoccupied space for randomly packed spheres to be 34% (Torquato, et al., 2000, Is Random Close Packing of Spheres Well Defined?, Phys. Lev. Lett. 84:2064-2067). Also, Torquato has shown that randomly packed oblate ellipsoids (like M&Ms) pack a little better, with an unoccupied space of about 27% (Donev, et al., 2004, Improving the Density of Jammed Disordered Packings Using Ellipsoids, Science 303:990). Thus, independent of particle size or shape, a storage matrix formed from closely packed, non porous, roughly-spherical objects presents about 25% to 35% of unoccupied interstitial void volume that can be used to sequester small molecules or biomolecules like DNA, RNA, or proteins in the dry state (see FIGS. 1 and 2).

Generally, in a matrix formed by closely packed spheres, the cross-sectional diameter of the interstitial spaces formed between nearly spherical particles is approximately equivalent to the radius of the surrounding particles. For example, if closely packed nanoparticles of about 50 nm in diameter are used to form the biomolecule storage matrix, the unoccupied volume between the nanoparticles in the dried phase will be characterized by a series of connected chambers with an average cross-sectional diameter in the 25 nm range, also the radius of the nanoparticles. A chamber of average cross-sectional diameter in the 25 nm range is of a size which is generally larger than the width of a DNA, RNA, or protein molecule, but much smaller than the size of a bacterium or mold spore. Thus, in a dried state formed by closest packing of 50 nm spheres, large biomolecules can be sequestered in the interstitial space between spheres, but biologically active agents, such as bacteria or mold, would be excluded and could not contaminate the matrix. Simple calculations indicate that 125 μg of such ceramic spheres in the fluid-free state will form a dry phase of approximately 10 mm² in cross section and will be about 1,000 spheres thick (FIG. 1).

Ceramic surfaces, including the spherical, non-porous ceramic nanoparticles of preferred embodiments of the present invention, present surface features that readily bind to nucleic acids and to proteins. For example, the literature cites many examples which use such ceramic particles for adsorption chromatography. To reduce or eliminate this intrinsic surface binding, the preferred embodiments of the present invention utilize surface passivation of nanoparticles with oxyanions to produce a coated surface with very low affinity for most biomolecules. The passivating coating of the nanoparticle allows a biomolecule to be retrieved from the nanoparticle matrix upon rehydration of the matrix without significant adsorptive loss on the nanoparticle surface. For example, one preferred embodiment is directed to the use of nanoparticles made of ZrO₂, a substance that ordinarily has a very high affinity for nucleic acids. However, as illustrated in the examples herein, when passivated by borate, ZrO₂ nanoparticles loose their affinity for nucleic acids and can be used directly as a dry state storage matrix for DNA. Other metal oxides, such as tungsten oxide, are known also to bind DNA and RNA and, when similarly passivated with borate, these nanoparticle materials can be used as well as a nanoparticle matrix for the dry state storage of DNA and other biomolecules.

The nanoparticle storage matrix can be modified by the selective addition of small molecule stabilizers. As part of an aqueous nanoparticle suspension, small molecule solutes can concentrate, upon drying, into the interstitial spaces between nanoparticles to form a paracrystalline state in direct contact with the biomolecule. The co-localization of the small molecule solutes and the biomolecules can provide additional stabilization of the biomolecule. The small molecule solutes can serve to inhibit the undesirable contact of the biomolecule with various contaminants or potential sources of degradation such as oxygen, free water, enzymes, or other reactive chemical species. (FIGS. 1 and 2).

One example of such a small stabilizing solute is boric acid (borate). Borate, as the Tris salt, is a standard component of biological buffers used for the analysis of DNA and RNA and is known to support a wide variety of biochemical analysis without causing functionally relevant alteration of DNA or RNA. In addition to its generally stabilizing effects on DNA, RNA, and protein structure, borate is useful as a small molecule component in the dry state nanoparticle storage matrix because it is known to inhibit microbial and fungal growth in the dry state. It is a good chelating agent and, therefore, inhibits metal-dependent reactions to DNA, RNA, and protein. Borate is strongly hydrated and can sequester free water molecules, thereby inhibiting undesired hydrolysis reactions that can occur upon DNA, RNA, or protein molecules.

Due to borate's ability to chemically stabilize DNA and RNA, it has been proposed by Brenner and colleagues that borate, as the parent mineral borax, was employed early in the process of chemical evolution as a method to stabilize RNA or DNA molecules that were created by pre-biotic chemical reactions (Ricardo, et al, 2004, Borate Minerals Stabilize Ribose, Science 303:196). As such, borate, as borax, may be viewed as the precursor to all known methods of nucleic acid stabilization in the dried or fluid state. More recently, borate at high concentration has been described as an effective method to solubilize, stabilize, and purify intact RNA molecules from complex plant sources that are known to be contaminated with a great deal of undesired RNAase activity (Wan and Wilkins, 1994, A modified hot borate method significantly enhances the yield of high-quality RNA from cotton, Anal. Bioch., 223:7-12).

In order to be a useful dry state storage technology, a matrix consisting of closely packed spheres should preferably satisfy a simple reversibility criterion. First, an aqueous suspension comprised of nanoparticles, small molecule stabilizers, and a biomolecule should preferably be able to air dry to form a solid state with sufficient mechanical integrity and flexibility that it will resist fragmentation during normal handling and storage. Upon rehydration, such a nanoparticle matrix should preferably be able to resuspend quickly to form a homogenous fluid phase that will liberate the sequestered biomolecule. Such a reversible process is preferred for the recovery of the stored biomolecule. For example, one preferred embodiment comprises an air-dried complex of about 120 μg of borate-passivated 25 nm diameter ZrO₂ nanoparticles mixed with about 40 μg of disodium tetraborate (borax) in which DNA is stored dry in the matrix in a mass range from about 1 ng to about 1 μg. Such a composition, and related modifications of such a composition, produce a nanoparticle storage complex that satisfies the desired reversibility characteristics described above.

In a particularly preferred embodiment, additional small molecules can be introduced as additives to the paracrystalline small molecule phase that forms within the interstitial spaces of the nanoparticle matrix, in order to improve the mechanical properties of the nanoparticle storage matrix and to facilitate its reversible dissociation upon re-hydration. One example of such small molecule additive is a plasticizer that can be used to improve the durability of the nanoparticle matrix and to facilitate the process of dissociation of the matrix once it is rehydrated. Such additives are chosen not to interfere with the chemical stability of the stored biomolecule and to resist microbial growth in the storage phase. A preferred set of such additives are polyols such as glycerol. Glycerol, when added to sodium borate at a mole ratio from about 0.5:1 up to about 2:1, improves the mechanical properties of the dried 25 my ZrO₂ nanoparticle plus borate storage matrix. Glycerol, like many vicinal poly-alcohols, is known to form a stable complex with borate and is sold commercially as the stable 2-1 glycerol borate complex. Glycerol is also a well-known plasticizer. Thus, in the present embodiment, via interaction with borate, a 0.5:1 to 2:1 mole ratio of glycerol is found to significantly improve the manufacturability of the nanoparticle matrix to render the dried matrix more resistant to vibrational damage, and to facilitate reversible dissociation of the air-dried storage matrix upon re-hydration.

Various embodiments of the reversible particle matrices are particularly useful for the storage of different types of biomolecules in different conditions. Generally, the particle matrices share the characteristic of being able to exist in both a fluid state and a dried state. For example, the particles can be in suspension in a fluid state; then the particles can be dried to a dry state; and the particles can easily be resuspended back into the fluid state. The ability of the particles to be in suspension or in a dried state, and the reversibility of these states, facilitates both the storage and the recovery of biomolecules.

One embodiment utilizes a particle storage matrix comprised of zirconium oxide nanoparticles passivated with borate. This embodiment is particularly useful for the storage of small quantities of pure or relatively pure nucleic acid samples such as DNA and RNA. The nanoparticles may be added directly to the sample or the nanoparticle matrix may be pre-dried, for example, into the wells of a 96-well or 384-well plate. One example of this embodiment uses zirconium oxide particles of about 20-40 nm in diameter which have been passivated so that the particles are substantially inert to the stored biomolecule. The storage matrix can be packaged as dry nanoparticle “dots” in a 96 well or 384 well format, or provided in a single tube format. The general model for use, which is described in greater detail in the examples, is to add up to about 100 μL of a dilute DNA sample, mix the sample with the particle matrix, and allow the sample to dry. Sample recovery is carried out by rehydrating the particle matrix. The sample is isolated from the particle matrix by centrifugation.

For storage of larger quantities of biomolecules, such as storage of microgram quantities of DNA, larger particles may be used for the storage matrix. For example, particles of about 200 nm in diameter may be used. The particles nanoparticles are passivated so they are substantially inert the stored biomolecule. The larger particle size accommodates the larger volume of biomolecule to be stored and allows for more rapid recovery of the biomolecule. The storage matrix can be packaged as dry nanoparticle “dots” in a 96 well or provided in a single tube format. Large sample sizes would be less amenable to a 384 well format,

For storage of crude samples, such as tissue samples and blood samples, it is often useful to digest the sample and to add the particle matrix directly to the tissue sample. For example, the sample may be digested with a protease such as savinase at 56° C. using a digestion buffer which may also serve also as part of the storage matrix. The digested sample may then be applied directly to dried particle matrix, or the particle matrix may be added to the sample in suspension. For crude sample storage, the particle matrix generally comprises of kaolin particles as described in greater detail in the examples.

For storage of other types of biomolecules such as proteins, serum proteins, antibodies, etc, the particles may be treated in various ways as described in greater detail in the examples. For example, for antibody storage, a thiophilic ligand may be used, for serum albumin, a Cibachrome blue coated nanoparticle may be used, etc.

The terms “biomolecules” and “biopolymers” are used interchangeable and are intended to include both short and long biopolymers including, but not limited to, such polymeric molecules as DNA, RNA, proteins, immunoglobulins, or carbohydrates. Thus, for example, the term includes both short (oligomeric) and long nucleic acid molecules, and similarly encompasses both small protein sequences (peptides) as well as longer polypeptides.

The term “ceramic” is defined as an inorganic crystalline molecular solid with non-metallic properties comprising of elements from Group I, Group II, Group III Transition Metals, Group IV, Group V, Group VI, and Group VII, and mixtures including such elements.

The term “nanoparticle” refers to a particle having an area to volume ratio of at least about 6 m²/cm³, and a sedimentation rate at one times gravitational force (1G) of at least about 6×10⁻⁴ cm/hr and not more than about 0.25 cm/hr. Such nanoparticles have a large surface area per unit volume or unit mass, thus offering a large surface area for manipulating a biopolymer.

The present invention contemplates particle matrices comprising a variety of substances of varying sizes depending on the application. Generally, materials that may be routinely obtained with a largest linear dimension of less than about 1 μm (“sub-micron”) are utilized. Nanoparticles comprising substances that are sufficiently robust, inert, and inexpensive are considered particularly useful. The present invention contemplates nanoparticles including metallic, semi-metallic, and non-metallic nanoparticles, including ceramics, clays, carbon-backboned or composite nanoparticles. Various embodiments utilize nanoparticles composed of phyllosilicate clay nanoparticles such as kaolin clay nanoparticles, zinc oxide nanoparticles, and tungsten oxide nanoparticles.

In one embodiment, the invention comprises solid-phase, non-porous particles for the manipulation of biomolecules, having a surface area to volume ratio (m²/cm³) greater than about 6 m²/cm³, a density (ρ) greater than about 2 gm/cm³, and sedimentation rates in water of Vₘᵢₙ greater than about 0.1 cm/min at 10,000 G and Vₘₐₓ, less than about 2 cm/minute at 500 G at standard temperature and pressure. In another embodiment, the particles have a density greater than about 2 gm/cm³ and less than or equal to about 2.5 gm/cm³ and effective spherical diameters, as determined by two times the Stokes radius, in the range of about 60 nm to about 1000 nm. In yet another embodiment, the particles have a density between about 2.5 gm/cm³ to about 3 gm/cm³ and effective spherical diameters, as determined by two times the Stokes radius, in the range of about 40 nm to about 800 nm. In another embodiment, the particles have a density between about 3 gm/cm³ to about 3.5 gm/cm³ and effective spherical diameters, as determined by two times the Stokes radius, of between about 35 nm to about 400 nm. In another embodiment, the particles have a density between about 3.5 gm/cm³ to about 4 gm/cm³ and effective spherical diameters, as determined by two times the Stokes radius, between about 20 nm to about 700 nm. In another embodiment, the particles have a density between about 4 gm/cm³ to about 4.5 gm/cm³ and effective spherical diameters, as determined by two times the Stokes radius, between about 30 nm to about 600 nm. In another embodiment, the particles have a density between about 4.5 gm/cm³ to about 5 gm/cm³ and effective spherical diameters, as determined by two times the Stokes radius, between about 25 nm to about 550 nm. In another embodiment, the particles have a density between about 5 gm/cm³ to about 5.5 gm/cm³ and effective spherical diameters, as determined by two times the Stokes radius, between about 25 nm to about 500 nm. In another embodiment, the particles have a density between about 5.5 gm/cm³ to about 6 gm/cm³ and effective spherical diameters, as determined by two times the Stokes radius, between about 25 nm to about 450 nm. In another embodiment, the particles have a density between about 6 gm/cm³ to about 6.5 gm/cm³ and effective spherical diameters, as determined by two times the Stokes radius, between about 20 nm to about 450 nm. In another embodiment, the particles have a density between about 6.5 gm/cm³ to about 7 gm/cm³ and effective spherical diameters as determined by two times the Stokes radius between about 20 nm to about 400 nm. In another embodiment, the particles have a density between about 7 gm/cm³ to about 7.5 gm/cm³ and effective spherical diameters, as determined by two times the Stokes radius, between about 20 nm to about 400 nm. In another embodiment, the particles have a density between about 7.5 gm/cm³ to about 14 gm/cm³ and effective spherical diameters, as determined by two times the Stokes radius, between about 15 nm to about 300 nm. In yet another embodiment, the particles have a density between about 14 gm/cm³ to about 20 gm/cm³ and effective spherical diameters, as determined by two times the Stokes radius, between about 12 nm to about 240 nm.

Preferably, when nanoparticles are used, the nanoparticles have a surface area per gram dry weight of about 6 m² per gram or greater, and an intrinsic density of greater than about 2 gm/cm³. Additional information regarding the properties of the preferred nanoparticles is found in co-pending U.S. patent application Ser. No. 11/338,124, filed on Jan. 23, 2006, and published as U.S. Patent Application Publication US 2006/0177855 A1 on Aug. 10, 2006 (incorporated by reference).

### EXAMPLES

The following examples are intended to illustrate, but not to limit, the invention in any manner, shape, or form, either explicitly or implicitly. While they are typical of those that might be used, other procedures, methodologies, or techniques known to those skilled in the art may alternatively be used.

### Example 1

### Washed Kaolin Nanoparticles

Washed kaolin nanoparticles were prepared for by first suspending the kaolin (CAS# 1332-58-7) nanoparticles, (Englehard, ASP ULTRAFINE), in N. AT, dimethyl formamide (DMF, CAS no. 68-12-2) at a ratio of 0.5 g to 1 g particles (dry weight) to 9 mL DMF. This colloidal suspension was incubated for a minimum of 16 hours. The nanoparticles were washed by a sedimentation-resuspension process by, first, sedimenting the nanoparticles out of suspension by centrifugation at 4000 G for 15 minutes; then resuspending the particles by adding 1 mL of liquid phase (water was used for this process) per 5 grams (dry weight) of particle-sediment, and mixing to form a thick slurry. Next, 9 mL of liquid phase (water) per gram (dry weight) was added to the slurry and mixed to form a confluent nanoparticle suspension. For the washed kaolin particles, for each 10 mL of the nanoparticle suspension, 1 mL of 5 M sodium chloride solution was added and mixed. The nanoparticle suspension was then incubated at room temperature for 12 to 16 hours. These particles were then washed again by the sedimentation-resuspension process, using water as the liquid phase, which was repeated three more times. The final concentration of particles in suspension was adjusted to 50 mg (dry weight) per milliliter in water.

### Example 2

### Borate Treated Kaolin Nanoparticles

The acid washed kaolin nanoparticles were prepared by first suspending the kaolin (CAS# 1332-58-7) nanoparticles, Englehardt, ASP G90 in de-ionized water at a weight to volume ratio of 1 to 3. This colloidal suspension was incubated for a minimum of 16 hours. The nanoparticles were then washed by a sedimentation-resuspension process. This process included sedimenting the nanoparticles out of suspension by centrifugation at 4000 G for 10 minutes, resuspending the kaolin in water at the same ratio, and repeating this process until the supernatant were clear with no sign of opalescence. The final kaolin pellet was resuspended at 1 to 3 ratio of weight per volume in water. Then an equal volume of 10% sulfuric acid was added to the suspension. This sulfuric acid/kaolin slurry was mixed and incubated at room temperature from 1 to 2 hours. Then the slurry was washed with distilled water by the sedimentation-resuspension process until the pH of the supernatant was the same as the pH of the distilled water. To this suspension, 1/50 volume of 500 mM NaF was added at a 1 to 10 ratio. The suspension was mixed and incubated. Then the suspension was subjected to one round of sedimentation-resuspension with distilled water, with the pellet being resuspended in 100 mM borate buffer (1:1 mixture of 100 mM boric acid to 100 mM sodium tetraborate) at ratio of 1 to 10 and mixed for at least 16 hours. This suspension was subjected to three rounds of sedimentation-resuspension with 10 mM borate buffer. The particles were stored in this condition until ready for dilution in 10 mM borate buffer.

### Example 3

### Borate Treated Aluminum Oxide

Aluminum oxide nanoparticles with a diameter range of 40 nm to 47 nm (Sigma-Aldrich catalog no. 544833) were suspend to a 1 to 10 ratio (weight to volume) in 50 mM HCl and incubate at room temperature for 1 hour under constant mixing. These particles were washed with distilled water by the sedimentation-resuspension process until the pH of the supernatant was the same as the pH of the distilled water. The nanoparticle pellet was resuspended in 100 mM borate buffer (1:1 mixture of 100 mM boric acid to 100 mM sodium tetraborate) at ratio of 1 to 10 and mixed for at least 16 hours. This suspension was subjected to three rounds of sedimentation-resuspension with 10 mM borate buffer. The particles were stored in this condition until ready for dilution in 10 mM borate buffer.

### Example 4

### Borate Treated Titanium Oxide

Titanium oxide nanoparticles with an average diameter of about 25 to 85 nm (Sigma-Aldrich catalog no. 634662) were was suspended at a 1 to 10 ratio (weight to volume) in 50 mM HCl and incubate at room temperature for 1 hour under constant mixing. These particles were washed with distilled water by the sedimentation-resuspension process until the pH of the supernatant was the same as the distilled water. The nanoparticle pellet was resuspended in 100 mM borate buffer (1:1100 mM boric acid to 100 mM sodium tetraborate) a ratio of 1 to 10 and mix for at least 16 hours. This suspension was subjected to three rounds of the sedimentation-resuspension with 10 mM borate buffer. The particles were stored in this condition until ready for dilution in 10 mM borate buffer.

### Example 5

### Borate Treated Tungsten Oxide

Tungsten oxide nanoparticles with an average diameter of about 25 nm (Sigma-Aldrich catalog no. 550086) were suspended at a 1 to 10 ratio (weight to volume) in 50 mM HCl and incubate at room temperature from 1 hour under constant mixing. These particles were washed with distilled water by the sedimentation-resuspension process until the pH of the supernatant was the same as the pH of the distilled water. The nanoparticle pellet was resuspended in 100 mM borate buffer (1:1 mixture of 100 mM boric acid to 100 mM sodium tetraborate) at a ratio of 1 to 10 and mixed for at least 16 hours. This suspension was subjected to three rounds of sedimentation-resuspension with 10 mM borate buffer. The particles were stored in this condition until ready for dilution in 10 mM borate buffer.

### Example 6

### Borate Treated Zirconium Oxide

Zirconium oxide nanoparticles with an average diameter of about 25 nm (Sigma-Aldrich catalog no. 544760) were suspended at a 1 to 10 ratio (weight to volume) in 50 mM HCl and incubate at room temperature from 1 hour under constant mixing. These particles were washed with distilled water by the sedimentation-resuspension process until the pH of the supernatant was the same as the pH of the distilled water. The nanoparticle pellet was resuspended in 100 mM borate buffer (1:1 mixture of 100 mM boric acid to 100 mM sodium tetraborate) at a ratio of 1 to 10 and mixed for at least 16 hours. This suspension was subjected to three rounds of sedimentation-resuspension with 10 mM borate buffer. The particles were stored in this condition until ready for dilution in 10 mM borate buffer.

### Example 7

### Borate-Passivated Zirconium Oxide as a Nanoparticle Storage Matrix

Zirconium oxide nanoparticles (having an average diameter of about 25 nm (and a full diameter range from about 20 nm to about 100 nm) were suspended at 10% by weight in 1 M HCl and incubated at room temperature for about 16 hours with constant mixing to passivate the particles. The particles were then centrifuged at 5,000 G to form a pellet and then resuspended in 0.1 M NaCl. The washing process was repeated until the resulting supernatant had a pH of 5 or greater. The particles were then resuspended to 10% by weight in 100 mM borax (disodium sodium tetraborate) and incubated for at least 16 hours. This suspension was subjected to sedimentation at 5000 G and then resuspended in 10 mM borax at a particle concentration of 6.25 mg/mL to be used as a stock solution.

### Example 8

### Borate-Passivated Tungsten Oxide as a Nanoparticle Storage Matrix

The process was the same as that described in Example 7 with the exception that the nanoparticle was composed of tungsten oxide having an average diameter of 25 nm, and a full diameter range from about 20 nm to about 100 nm.

### Example 9

### ZrO₂ Nanoparticle Storage Matrix in a Microtiter Plate

A nanoparticle storage matrix was fabricated in a round bottom, polypropylene, 96 well microtiter plate. 20 μL of total fluid was added per well composed of 125 fig of the ZrO₂ nanoparticles of Example 7 and 40 μg of borax. The added 20 μL suspension was air dried at room temperature onto the bottom surface of each well to form a discrete pellet of the storage matrix. The drying process typically required 16 hours of incubation.

### Example 10

### WO₃ Nanoparticle Storage Matrix in a Microtiter Plate

The process was the same as that described in Example 9 with the exception that the nanoparticle component is tungsten oxide as described in Example 8. The 20 μL suspension was air dried at room temperature onto the bottom surface of each well of a 96 well microtiter plate for at least 16 hours.

### Example 11

### ZrO₂ Nanoparticle Storage Matrix with Glycerol

The process is the same as described in Example 9 with the addition of glycerol at a total mass of 55 μg per 20 μL of total suspension. The mixed suspension was air dried at room temperature onto the bottom surface of each well for at least 16 hours.

### Example 12

### ZrO₂ Nanoparticle Storage Matrix with Glycerol

The process was the same as described in Example 11 with the exception that glycerol was added to 110 μg per 20 μL of total suspension. The mixed suspension was air dried at room temperature onto the bottom surface of each well for at least 16 hours.

### Example 13

### ZrO₂ Nanoparticle Storage Matrix with Glycerol

The process was the same as described in Example 11 with the exception that glycerol was added to 165 μg per 20 μL. The mixed suspension was air dried onto the bottom surface of each well for at least 16 hours at room temperature.

### Example 14

### Storage of Pure DNA in an Air Dried Nanoparticle Matrix in a Microtiter Plate

To the air-dried nanoparticle pellets described in Example 9, Example 10, Example 11, Example 12, and Example 13, a solution of human DNA was added in TE buffer (Roche Gen) at a volume of 20 μL, per well. The dried nanoparticle matrix of each well was resuspended in this DNA solution by pipetting to confluence. The resulting suspension was then air-dried back to a dried nanoparticle pellet in the same well. The amount of DNA added to each well ranged from about 1 ng to about 30 ng. After the air-dried nanoparticle matrix was formed with the DNA sample, the plate was stored at either room temperature, (approximately 25° C.) or at 56° C. for up to 24 days.

### Example 15

### Recovery of Pure DNA from the Air Dried Nanoparticle Matrix by Resuspension in Water

To retrieve the DNA from the air-dried nanoparticle matrix of Example 14, 20 μL of water was added to each well and incubated at room temperature for about 15 minutes. The matrix was dissociated by repeated pipetting until homogenous. The resulting nanoparticle suspension was then incubated at either 56° C. or at room temperature for a minimum of 30 minutes, with occasional pipetting or vortex mixing to ensure a confluent nanoparticle suspension. After that incubation, the suspension was subjected to centrifugation at 8,000 G or greater for 3 minutes to pellet the spent nanoparticles. The DNA-containing supernatant above the spent nanoparticle pellet was retrieved by pipetting and then used “as is” or diluted for further analysis.

### Example 16

### PCR Analysis of Human DNA

PCR was used to compare and evaluate the DNA capture process using various nanoparticles as described in the Examples. These PCR analyses were based on a nuclear chromosome encoded gene, amelogenin, encoded on both the X and Y chromosomes.

The primers used were of two sequences. The sequence of the first primer was 5′-AGA TGA AGA ATG TGT GTG ATG GAT GTA-3′ (SEQ ID NO: 1), and the sequence of the second primer was 5′-GGG CTC GTA ACC ATA GGA AGG GTA-3′ (SEQ ID NO:2). Both sequences were derived from the amelogenin sequence in GenBank with accession number AY040206. The PCR product from these two primers is a 558 base pair long fragment. In general, PCR reactions were carried out as follows. The PCR reactions were carried out in a 50 μL volume. The reactions contained 1× Roche PCR Buffer, 1.5 mM MgCl₂, 0.4 μM primers, 0.2 mM dNTPs, 0.16 mg/ml BSA, and 0.4 of Fast Start Taq at 5 U/μL. The conditions for these PCR tests were as follows. The first step was at 94° C. for 4 minutes. Then there were 35 cycles composed of three steps including 94° C. for 1 minute, followed by 65° C. for 1 minute, and then 72° C. for 1 minute. After these 35 cycles, the reactions were incubated at 72° C. for 7 minutes followed by a holding step at 15° C. until the reactions were stopped. All of PCR results were evaluated by electrophoresis of ⅕ of the reaction volume in agarose gels using a Tris-Borate-EDTA buffer system. The molecular weight control used was the 1 Kb DNA ladder from Invitrogen (catalog no. 15615-016). The PCR controls used were a negative control, a reaction with no added DNA template, and four positive controls with a fixed and known amount of human DNA (Roche Human Genomic DNA catalog no. 1691112) used as PCR templates, generally at concentrations of 10 ng, 1 ng, 0.1 ng and 0.01 ng per 50 μL PCR reaction.

### Example 17

### Recovery of Pure DNA from Dry State Storage for 1 Day and 3 Days, Nanoparticle Matrix Composed of WO₃ Passivated with Borax

This example is a PCR analysis of DNA recovered from dry state storage for 1 day and 3 days. The nanoparticle matrix was composed of WO₃ passivated with borax as described in Example 10. PCR assays were done in a 25 μL volume, using 4 μL of DNA as template for all reactions. All template DNA samples were adjusted to a dilution of 0.05 ng per microliter (i.e. 0.2 ng per reaction), assuming 100% recovery of the original input DNA. The PCR target sequence was the human amelogenin locus which yields a 558 bp amplicon and is described in detail above in Example 16. The PCR product was analyzed by 2% agarose electrophoresis with Tris-borate buffer at 150 volts for 45 minutes.

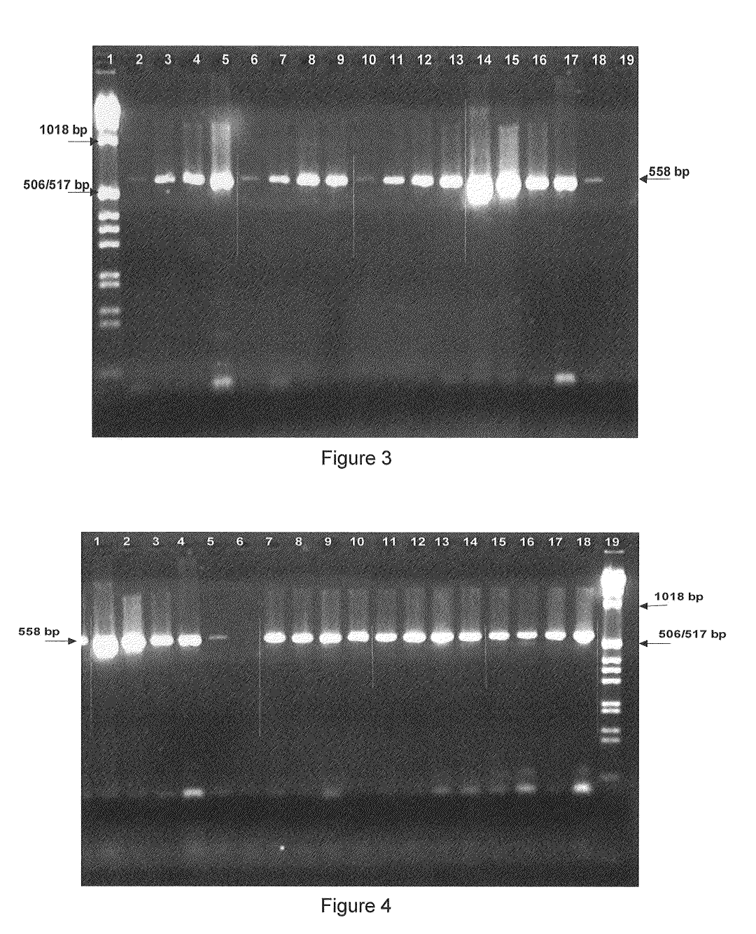

The results are shown in FIG. 3 and demonstrate the reversible DNA recovery from a WO₃ nanoparticle matrix passivated with borax for up to three days of room temperature dry state storage. Referring to FIG. 3, lane 1 contains a double-stranded DNA molecular weight marker. Lanes 2-5 contain DNA samples extracted after one hour as a nanoparticle slurry. Lanes 6-9 contain a DNA samples extracted after one day of dry-state storage within a WO₃/borax nanoparticle matrix. Lanes 10-13 contain a DNA samples extracted after three days dry-state storage within a WO₃/borax nanoparticle matrix. Lanes 14-18 contain semi-quantitative-PCR controls using various known amounts of human DNA, and lane 19 is the negative PCR control, with no DNA in the assay.

More specifically, in FIG. 3, Lane 2 contains a DNA sample of 1 ng extracted after one hour as a nanoparticle slurry. Lane 3 contains a DNA sample of 3 ng extracted after one hour as a nanoparticle slurry. Lane 4 contains a DNA sample of 10 ng extracted after one hour as a nanoparticle slurry. Lane 5 contains a DNA sample of 30 ng extracted after one hour as a nanoparticle slurry. Lane 6 contains a DNA sample of 1 ng extracted after one day of dry-state storage within a WO₃/borax nanoparticle matrix. Lane 7 contains a DNA sample of 3 ng extracted after one day dry-state storage within a WO₃/borax nanoparticle matrix. Lane 8 contains a DNA sample of 10 ng extracted after one day dry-state storage within a WO₃/borax nanoparticle matrix. Lane 9 contains a DNA sample of 30 ng extracted after one day dry-state storage within a WO₃/borax nanoparticle matrix. Lane 10 contains a DNA sample of 1 ng extracted after three days dry-state storage within a WO₃/borax nanoparticle matrix. Lane 11 contains a DNA sample of 3 ng extracted after three days dry-state storage within a WO₃/borax nanoparticle matrix. Lane 12 contains a DNA sample of 10 ng extracted after three days dry-state storage within a WO₃/borax nanoparticle matrix. Lane 13 contains a DNA sample of 30 ng extracted after three days dry-state storage within a WO₃/borax nanoparticle matrix. Lane 14 contains a semi-quantitative-PCR control of 10 ng of human DNA input per assay. Lane 15 is a semi-quantitative-PCR control of 1.0 ng of human DNA input per assay. Lane 16 is a semi-quantitative-PCR control of 0.2 ng of human DNA input per assay. Lane 17 is a semi-quantitative-PCR control of 0.10 ng of human DNA input per assay. Lane 18 is a semi-quantitative-PCR control of 0.001 ng of human DNA input per assay. Lane 19 is a negative PCR control in which no human DNA was added per assay.

### Example 18

### Recovery of Pure DNA from Dry State Storage for 1 Day and 3 Days, Nanoparticle Matrix Composed of ZrO₉ Passivated with Borax

This example is a PCR analysis of DNA recovered from dry state storage for 1 day and 3 days using a nanoparticle matrix composed of ZrO₂ passivated with borax as described in Example 9. PCR assays were done in a 25 μL volume, using 4 μL of DNA as template for all reactions. All template DNA samples were adjusted to a dilution of 0.05 ng per microliter (i.e. 0.2 ng per reaction), assuming 100% recovery of the original input DNA. The PCR target sequence was the human amelogenin locus which yields a 558 bp amplicon and is described in detail above in Example 16. The PCR product was analyzed by 2% agarose electrophoresis with Tris-borate buffer at 150 volts for 45 minutes.

The results are shown in FIG. 4 and demonstrate the reversible DNA recovery from a ZrO₂ nanoparticle matrix passivated with borax for up to three days of room temperature dry state storage. The data also show that, at the formulations used, recovery with the ZrO₂ nanoparticle matrix was more efficient than recovery with the WO₃ nanoparticle matrix of Example 61. Referring to FIG. 4, lanes 1-5 are semi-quantitative PCR controls using known amounts of human DNA. Lane 6 is a negative PCR control in which no human DNA was added. Lanes 7-10 contain DNA samples extracted after one hour as a nanoparticle slurry. Lanes II-14 contain DNA samples extracted after one day dry-state storage within a ZrO₂/borax nanoparticle matrix. Lanes 15-18 contain a DNA samples extracted after three days dry-state storage within a ZrO₂/borax nanoparticle matrix. Lane 19 is a double-stranded DNA molecular weight marker.

More specifically, in FIG. 4, lane 1 is a semi-quantitative PCR control using 10 ng of human DNA input per assay. Lane 2 is a semi-quantitative PCR control using 1.0 ng of human DNA input per assay. Lane 3 is a semi-quantitative PCR control using 0.2 ng of human DNA input per assay. Lane 4 is a semi-quantitative PCR control using 0.10 ng of human DNA input per assay. Lane 5 is a semi-quantitative PCR control using 0.001 ng of human DNA input per assay. Lane 6 is a negative PCR control in which no human DNA was added per assay. Lane 7 contains a DNA sample of 1 ng extracted after one hour as a nanoparticle slurry. Lane 8 contains a DNA sample of 3 ng extracted after one hour as a nanoparticle slurry. Lane 9 contains a DNA sample of 10 ng extracted after one hour as a nanoparticle slurry. Lane 10 contains a DNA sample of 30 ng extracted after one hour as a nanoparticle slurry. Lane 11 contains a DNA sample of 1 ng extracted after one day dry-state storage within a ZrO₂/borax nanoparticle matrix. Lane 12 contains a DNA sample of 3 ng extracted after one day dry-state storage within a ZrO₂/borax nanoparticle matrix. Lane 13 contains a DNA sample of 10 ng extracted after one day dry-state storage within a ZrO₂/borax nanoparticle matrix. Lane 14 contains a DNA sample of 30 ng extracted after one day dry-state storage within a ZrO₂/borax nanoparticle matrix. Lane 15 contains a DNA sample of 1 ng extracted after three days dry-state storage within a ZrO₂/borax nanoparticle matrix. Lane 16 contains a DNA sample of 3 ng extracted after three days dry-state storage within a ZrO₂/borax nanoparticle matrix. Lane 17 contains a DNA sample of 10 ng extracted after three days dry-state storage within a ZrO₂/borax nanoparticle matrix. Lane 18 contains a DNA sample of 30 ng extracted after three days dry-state storage within a ZrO₂/borax nanoparticle matrix. Lane 19 is a double-stranded DNA molecular weight marker.

### Example 19

### Comparison of Dry-State Storage of Pure DNA for 10 or 34 days using Nanoparticles Composed of Zirconium Oxide or Tungsten Oxide

This example compares the dry-state storage of DNA for 10 and 34 days using either nanoparticles composed of zirconium oxide prepared according to Example 9 or nanoparticles composed of tungsten oxide prepared according to Example 10.

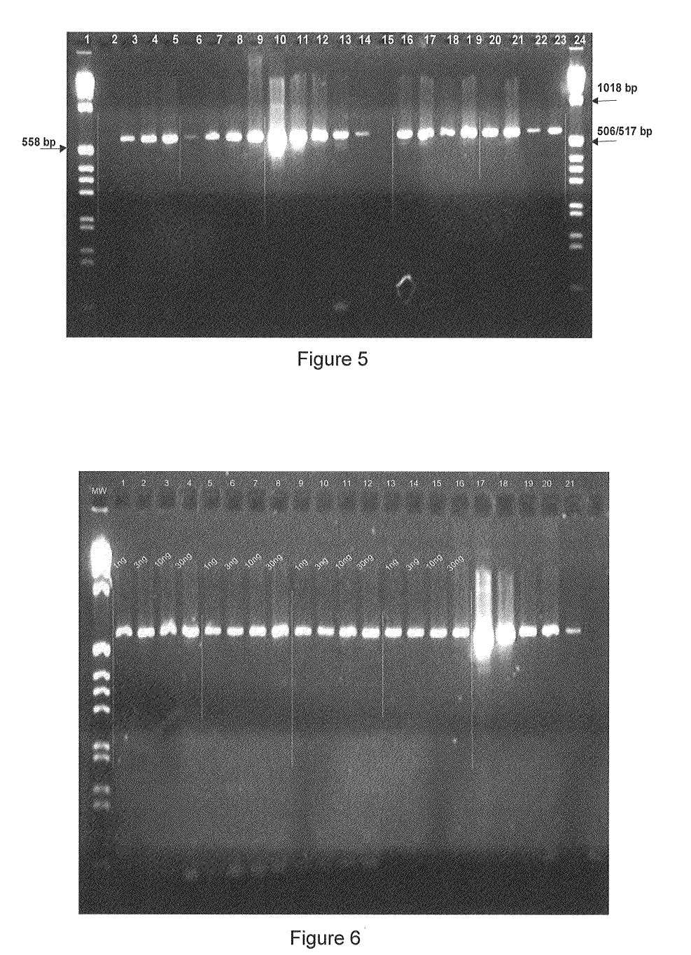

PCR assays were done in a 25 μL volume, using 4 μL of DNA as template for all reactions. All template DNA samples were adjusted to a dilution of 0.05 ng per microliter (i.e. 0.2 ng per reaction), assuming 100% recovery of the original input DNA. The PCR target sequence was the human amelogenin locus which yields a 558 bp amplicon and is described in detail above in Example 16. The PCR product was analyzed by 2% agarose electrophoresis with Tris-borate buffer at 150 volts for 45 minutes. As seen in FIG. 5, data demonstrate that recovery with the ZrO₂ nanoparticle matrix remains more efficient than recovery with the WO₃ nanoparticle matrix after 10 or 34 days of dry state storage.

Referring to FIG. 5, lane 1 is a double-stranded DNA molecular weight marker. Lanes 2-5 contain DNA samples extracted after ten days dry-state storage within a WO₃ nanoparticle matrix. Lanes 6-9 contain DNA samples of 1 ng extracted after thirty four days dry-state storage within WO₃ nanoparticle matrix. Lanes 10-14 are semi-quantitative PCR controls using known quantities of human DNA. Lane 15 is a negative PCR control in which no human DNA was added. Lanes 16-19 contain DNA samples of 1 ng extracted after ten days dry-state storage within ZrO₂ nanoparticle matrix. Lanes 20-23 contain DNA samples extracted after thirty four days dry-state storage within ZrO₂ nanoparticle matrix.

More specifically, in FIG. 16, lane 1 is a double-stranded DNA molecular weight marker. Lane 2 contains a DNA sample of 1 ng extracted after ten days dry-state storage within a WO₃ nanoparticle matrix. Lane 3 contains a DNA sample of 3 ng extracted after ten days dry-state storage within WO₃ nanoparticle matrix. Lane 4 contains a DNA sample of 10 ng extracted after ten days dry-state storage within WO₃ nanoparticle matrix. Lane 5 contains a DNA sample of 30 ng extracted after ten days dry-state storage within WO₃ nanoparticle matrix. Lane 6 contains a DNA sample of 1 ng extracted after thirty four days dry-state storage within WO₃ nanoparticle matrix. Lane 7 contains a DNA sample of 3 ng extracted after thirty four days dry-state storage within WO₃ nanoparticle matrix. Lane 8 contains a DNA sample of 10 ng extracted after thirty four days dry-state storage within WO₃ nanoparticle matrix. Lane 9 contains a DNA sample of 30 ng extracted after thirty four days dry-state storage within WO₃ nanoparticle matrix. Lane 10 is a semi-quantitative PCR control of 10 ng of human DNA input per assay. Lane 11 is a semi-quantitative PCR control of 1.0 ng of human DNA input per assay. Lane 12 is a semi-quantitative PCR control of 0.2 ng of human DNA input per assay. Lane 13 is a semi-quantitative PCR control of 0.10 ng of human DNA input per assay. Lane 14 is a semi-quantitative PCR control of 0.001 ng of human DNA input per assay. Lane 15 is a negative PCR control in which no human DNA input was added per assay. Lane 16 contains a DNA sample of 1 ng extracted after ten days dry-state storage within ZrO₂ nanoparticle matrix. Lane 17 contains a DNA sample of 3 ng extracted after ten days dry-state storage within ZrO₂ nanoparticle matrix. Lane 18 contains a DNA sample of 10 ng extracted after ten days dry-state storage within ZrO₂ nanoparticle matrix. Lane 19 contains a DNA sample of 30 ng extracted after ten days dry-state storage within ZrO₂ nanoparticle matrix. Lane 20 contains a DNA sample of 1 ng extracted after thirty four days dry-state storage within ZrO₂ nanoparticle matrix. Lane 21 contains a DNA sample of 3 ng extracted after thirty four days dry-state storage within ZrO₂ nanoparticle matrix. Lane 22 contains a DNA sample of 10 ng extracted after thirty four days dry-state storage within ZrO₂ nanoparticle matrix. Lane 23 contains a DNA sample of 30 ng extracted after thirty fours day dry-state storage within ZrO₂ nanoparticle matrix.

### Example 20

### Recovery of Pure DNA from 7 Days of Dry State Storage using a Nanoparticle Matrix Composed of ZrO₂ Passivated with Borax

This experiment analyzed DNA recovered from 7 days of dry storage using two different nanoparticle matrices, each composed of ZrO₂ passivated with borax, and each with the addition of glycerol as a plasticizer, as described in Examples 11 and 12. Storage conditions were tested with each type of nanoparticle at two storage temperatures, room temperature and 56° C.

In this experiment, different storage conditions were evaluated. All nanoparticle slurry samples were dried at room temperature for 16 hours. The two types of nanoparticles used were ZrO₂ nanoparticles with borax and with differing amounts of added glycerol. The nanoparticles of Example 11 were used for samples 1-8 and the nanoparticles of Example 12 were used for samples 9-16. Two different post drying storage conditions were also tested. Samples 5-8 and 13-16 were stored at room temperature, and samples 1-4 and samples 9-12 were stored at 56° C. Elution conditions were at room temperature for 60 minutes followed by 56° C. for 30 minutes. All samples were eluted in 20 μL of water. For each sample, the amount tested was adjusted to a DNA input of 0.2 ng, assuming 100% recovery of the DNA.

PCR assays were done in a 25 μL volume, using 4 μL of DNA as template for all reactions. All template DNA samples were adjusted to a dilution of 0.05 ng per microliter (i.e. 0.2 ng per reaction), assuming 100% recovery of the original input DNA. The PCR target sequence was the human amelogenin locus which yields a 558 bp amplicon as described in detail above in Example 6. The PCR product was analyzed by 2% agarose electrophoresis with Tris-borate buffer at 150 volts for 45 minutes.

Referring to FIG. 6, PCR assays of the 1 ng samples, in lanes 1, 5, 9, and 13, tested 4 μL of the 20 μL eluate. PCR assays of the 3 ng samples, in lanes 2, 6, 10, and 14, tested 4 μL of the ⅓ dilution of the 20 μL eluate. PCR assay of the 10 ng samples, in lanes 3, 7, 11, and 15, tested 4 μL of the 1/10 dilution of the 20 μL eluate. PCR assays of the 30 ng samples, in lanes 4, 8, 12, and 16 tested 4 NIL of the 1/30 dilution of the 20 μL eluate. Lanes 17-21 are PCR controls containing known amounts of human DNA with 10 ng in lane 17, 1.0 ng in lane 18, 0.2 ng in lane 19, 0.1 ng in lane 20, and 0.01 ng in lane 21. The lane marked MW contains a double-stranded DNA molecular weight marker, and lane 22 contains a negative PCR control with no DNA.

As seen in FIG. 6, these data demonstrate reversible DNA recovery from a ZrO₂ nanoparticle matrix passivated with borax, with the addition of glycerol as a plasticizer at two plasticizer amounts, out to 7 days of room temperature dry state storage. The data show that, with both of the nanoparticle formulations (Examples 56 and 57), recovery with the ZrO₂ nanoparticle matrix, with glycerol as an additive, approached 100%.

### Example 21

### Recovery of Pure DNA from 10 Days of Dry State Storage, Nanoparticle Matrix Composed of ZrO₂, Passivated with Borax

This experiment is a PCR analysis of DNA recovered after 10 days of dry state storage using a nanoparticle matrix composed of ZrO₂ passivated with borax as described in Examples 11 and 12. PCR assays were done in a 25 μL volume, using 4 μL of DNA as template for all reactions. All template DNA samples were adjusted to a dilution of 0.05 ng per microliter (i.e. 0.2 ng per reaction), assuming 100% recovery of the original input DNA. The PCR target sequence was the human amelogenin locus which yields a 558 bp amplicon and is described in detail in Example 16. The PCR product was analyzed by 2% agarose electrophoresis with Tris-borate buffer at 150 volts for 45 minutes.

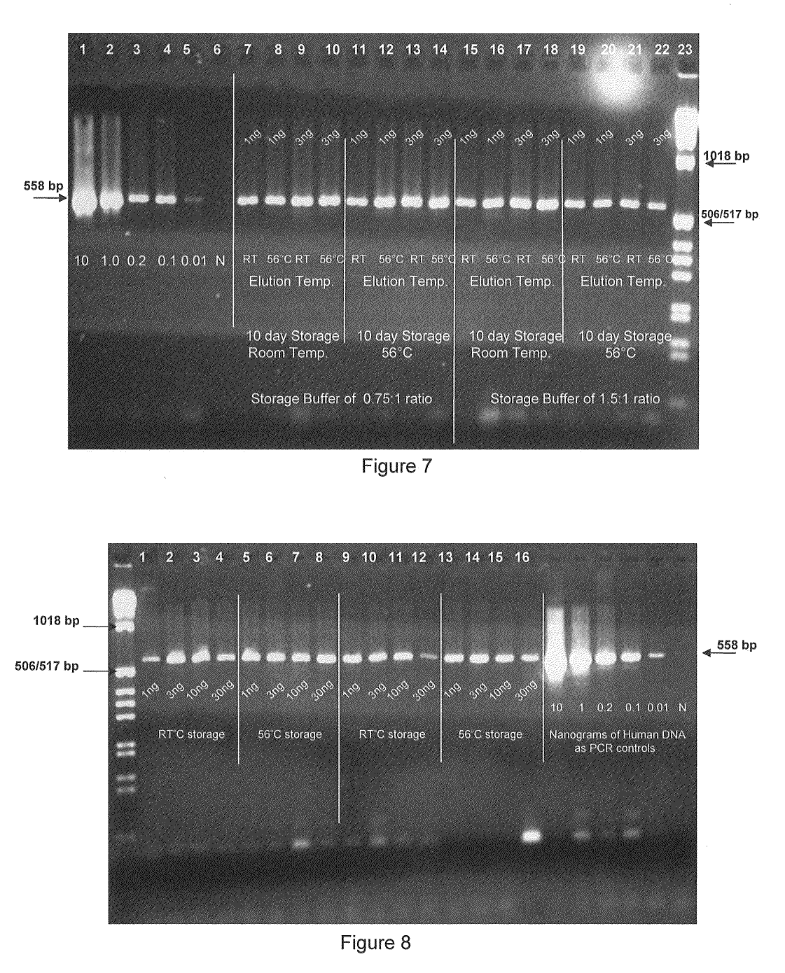

As seen in FIG. 7, these data demonstrate reversible DNA recovery from a ZrO₂ nanoparticle matrix passivated with borax with the addition of glycerol as a plasticizer at two plasticizer amounts out to 10 days of room temperature dry state storage. The nanoparticle matrix of Example 11 used a 0.75:1 glycerol-borate storage buffer, and the nanoparticle matrix of Example 12 used a 1.5:1 glycerol-borate storage buffer. The data also show that, for both of the nanoparticle formulations used, recovery with the ZrO₂ nanoparticle matrix, with glycerol as an additive, approached 100%. The data also show that the DNA the quality and the quantity of the recovered DNA, as a PCR template, do not appear to be affected by raising the storage temperature to 56° C.

Referring to FIG. 7, lanes 1-5 are PCR controls containing known amounts of human DNA with 10 ng in lane 1, 1.0 ng in lane 2, 0.2 ng in lane 3, 0.1 ng in lane 4, and 0.01 ng in lane 5. Lane 6 is a negative PCR control with no DNA. Lanes 7-14 contain PCR products from DNA extracted after 10 days dry storage using the nanoparticles of Example 11. Lanes 7-10 show the products from DNA stored at room temperature, and lanes 11-14 show the products from DNA stored at 56°. Lanes 15-22 contain PCR products from DNA extracted after 10 days dry storage using the nanoparticles of Example 12. Lanes 15-18 show the products from DNA stored at room temperature, and lanes 19-22 show the products from DNA stored at 56°. Lanes 7, 9, 11, 13, 15, 17, 19, and 21 contain the products from DNA eluted at room temperature, and lanes 8, 10, 12, 14, 16, 18, 20, and 22 contain the products from DNA eluted at 56° C. Additionally, lanes 7, 8, 11, 12, 15, 16, 19, and 20 are PCR assays using 1 ng of DNA, and lanes 9, 10, 13, 14, 17, 18, 21, and 22 are PCR assays using 3 ng of DNA. Lane 23 is a double-stranded DNA molecular weight marker.

### Example 22

### Recovery of Pure DNA from 12 Days of Dry State Storage, Nanoparticle Matrix Composed of ZrO₂ Passivated with Borax

This experiment is a PCR analysis of DNA recovered after 12 days of dry state storage using a nanoparticle matrix composed of ZrO₂ passivated with borax. PCR assays were done in a 25 μL volume, using 4 μL of DNA as template for all reactions. All template DNA samples were adjusted to a dilution of 0.05 ng per microliter (i.e. 0.2 ng per reaction), assuming 100% recovery of the original input DNA. The PCR target sequence was the human amelogenin locus which yields a 558 bp amplicon and is described in detail in Example 16. The PCR product was analyzed by 2% agarose electrophoresis with Tris-borate buffer at 150 volts for 45 minutes.

This Example compares storage of DNA for 12 days at room temperature and at 56° C. This example and also compares two processes for delivering the nanoparticle matrix into the microtiter plate, either predried in the microtiter plate or added directly as a slurry to the sample. The nanoparticle storage buffer used was 0.75:1 glycerol to borate, as used in Example 11. PCR assays were initiated with the DNA directly from the eluate or from eluate dilutions adjusted to provide approximately 0.2 ng of DNA per reaction.

The results are shown in FIG. 8. For the 1 ng DNA samples (in lanes 1, 5, 9, and 13), PCR was initiated with 4 μL of the eluate, directly. For the 3 ng DNA samples (in lanes 2, 6, 10, and 14), PCR was initiated with 4 μL of a ⅓ dilution of the eluate. For the 10 ng DNA samples (in lanes 3, 7, 10, and 15), PCR was initiated with 4 μL, of a 1/10 dilution of the eluate. For the 30 ng DNA samples (in lanes 4, 8, 11, and 16), PCR was initiated with 4 μL of a 1/30 dilution of the eluate. Lanes 1-8 show the product of the DNA sample added to the nanoparticle matrix that was pre-dried in the micro-titer plate. Then the sample and the nanoparticle matrix were mixed together into a slurry, and then re-dried in the plate. Lanes 9-16 show the product of the DNA sample was added and mixed with a slurry of the nanoparticle matrix and then dried in the plate. The data show that DNA recovery and the quality of the recovered DNA, as a PCR template, were comparable with the storage temperature at room temperature of at 56° C.

### Example 23

### Recovery of Pure DNA from 25 Days of Dry State Storage using a Nanoparticle Matrix Composed of ZrO₂ Passivated with Borax

The experiment is a PCR analysis of DNA recovered from 25 days of dry state storage using a nanoparticle matrix composed of ZrO₂ passivated with borax as described in Example 11. PCR assays were done in a 25 μL volume, using 4 μL of DNA as template for all reactions. All template DNA samples were adjusted to a dilution of 0.05 ng per microliter (i.e. 0.2 ng per reaction), assuming 100% recovery of the original input DNA. The PCR target sequence was the human amelogenin locus which yields a 558 bp amplicon and is described in detail in Example 16. The PCR product was analyzed by 2% agarose electrophoresis with Tris-borate buffer at 150 volts for 45 minutes.

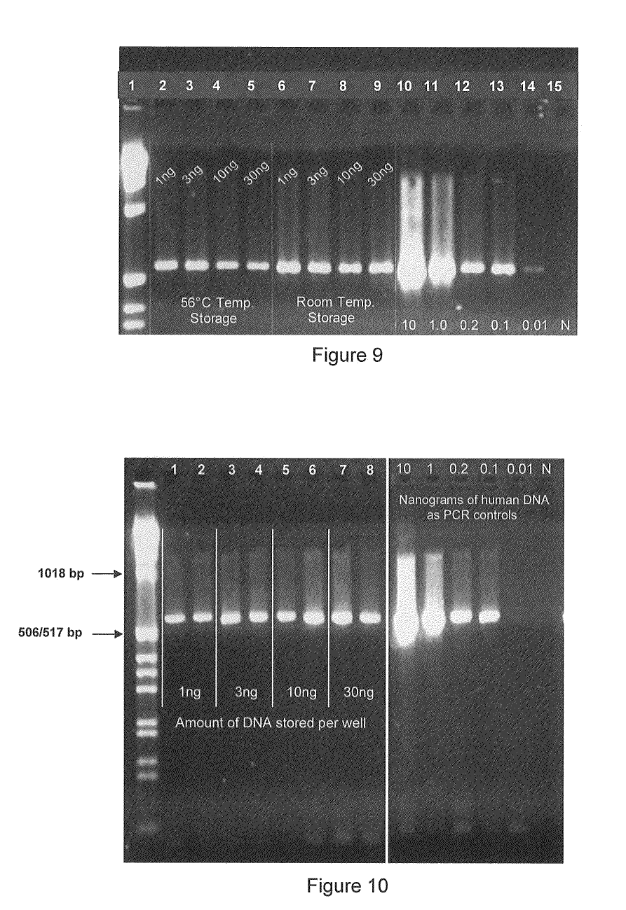

Referring to FIG. 9, Lane 1 contains a double stranded DNA molecular weight marker. Lanes 2-9 contain PCR products from DNA extracted after 25 days dry storage using the nanoparticles of Example 11. The products in lanes 2-5 are the result of storage at 56° C., and the products in lanes 6-9 are the results of storage at room temperature. Lanes 2, 4, 6, and 8 contain the PCR products from 1 ng of extracted DNA, and lanes 3, 5, 7, and 9 contain the PCR products from 3 ng of extracted DNA. Lanes 10-14 are PCR controls containing known amounts of human DNA with 10 ng in lane 10, 1.0 ng in lane 11, 0.2 ng in lane 12, 0.1 ng in lane 13 and 0.01 ng in lane 14. Lane 15 is a negative PCR control with no DNA added.

As seen in FIG. 9, these data demonstrate reversible DNA recovery from a ZrO₂ nanoparticle matrix passivated with borax with the addition of glycerol as a plasticizer, as in Example 11, out to 25 days of room temperature dry state storage. The data also show that, with the nanoparticle formulation, use, recovery with the ZrO₂ nanoparticle matrix, with the addition of glycerol, approached 100%. The data show that DNA recovery and the quality of the recovered DNA, as a PCR template, were not affected by raising the storage temperature to 560 C.

### Example 24

### Recovery of Pure DNA from 52 Days of Dry State Storage at Room Temperature using a Nanoparticle Matrix Composed of ZrO₂ Passivated with Borax

The experiment is a PCR analysis of DNA recovered from 52 days of dry state storage at room temperature using a nanoparticle matrix composed of ZrO₂ passivated with borax in a storage buffer of 1.5 to 1 glycerol to borate. All template DNA samples were adjusted to approximately 0.2 ng of DNA per reaction, assuming 100% recovery of the original input DNA. The PCR target sequence was the human amelogenin locus which yields a 558 bp amplicon and is described in detail in Example 16. The PCR product was analyzed by 2% agarose electrophoresis with Tris-borate buffer at 150 volts for 45 minutes.

The results are shown in FIG. 10. Varying amounts of DNA were stored in the nanoparticle matrix. Lanes 1 and 2 show the results for the storage of 1 ng of DNA; lanes 3 and 4 show the results for the storage of 3 ng of DNA; lanes 5 and 6 show the results for the storage of 10 ng of DNA; and lanes 7 and 8 show the results for the storage of 30 ng of DNA.

### Example 25

### Recovery of Pure DNA from 72 Days of Dry State Storage using a Nanoparticle Matrix Composed of ZrO₂ Passivated with Borax

This experiment is a PCR analysis of DNA recovered from 72 days of dry state storage using a nanoparticle matrix composed of ZrO₂ passivated with borax. The samples were stored under two temperature conditions, room temperature and 56° C.

Template DNA samples were adjusted to provide approximately 0.2 ng of DNA per reaction, assuming 100% recovery of the original input DNA. The PCR target sequence was the human amelogenin locus which yields a 558 bp amplicon and is described in detail in Example 16. The 56° C. storage temperature is used for accelerated stability testing of DNA storage. Standard calculations predict an approximate 4-fold increase in the degradation rate of the DNA for each 10° C. increase in temperature. In this case, a factor of about 64 for the 30° C. difference between storage at room temperature and 56° C. In other words, storage for 72 days at 56° C. should be roughly equivalent to storage for 4,608 days (12.6 years) at room temperature.

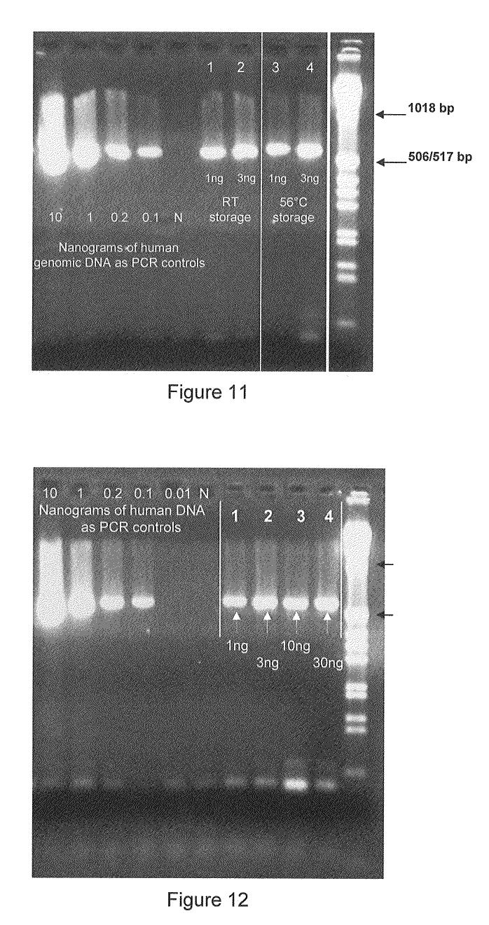

The results are shown in FIG. 11. Lane 1 contains 4 μL, of a 20 μL elution of a 1 ng DNA sample stored at room temperature for 72 days. Lane 2 contains 1.3 μL of a 20 μL elution of a 3 ng DNA sample stored at room temperature for 72 days. Lane 3 contains 4 μL of a 20 μL elution of a 1 ng DNA sample stored at 56° C. for 72 days. Lane 4 contains 1.3 μL of a 20 μL elution of a 3 ng DNA sample stored at 56° C. for 72 days. The data show that DNA recovery and the quality of the recovered DNA, as a PCR template, were not affected by raising the storage temperature to 56° C. for 72 days.

### Example 26

### Recovery of Pure DNA from 72 Days of Dry State Storage at 56° C. using a Nanoparticle Matrix Composed of ZrO₂ Passivated with Borax

This experiment is a PCR analysis of DNA recovered from 72 days of dry state storage using a nanoparticle matrix composed of ZrO₂ passivated with borax. The samples were stored at 56° C. As explained above, storage for 72 days at 56° C. should be roughly equivalent to storage for 4,608 days (12.6 years) at room temperature. The nanoparticle storage buffer contained 1.5:1 glycerol to borate. Template DNA samples were adjusted to provide approximately 0.2 ng of DNA per reaction, assuming 100% recovery of the original input DNA. The PCR target sequence was the human amelogenin locus which yields a 558 bp amplicon and is described in detail in Example 16.

The results are shown in FIG. 12. Lane 1 contains 4 μL of a 20 μL elution of a 1 ng DNA sample stored at room temperature for 72 days. Lane 2 contains 4 μL of a 1:2 dilution of a 20 uL elution of a 3 ng DNA sample stored at 56° C. for 72 days. Lane 3 contains 4 μL of a 1:10 dilution of a 20 μL elution of a 10 ng DNA sample stored at 56° C. for 72 days. Lane 4 contains 4 μL, of a 1:30 dilution of a 20 μL, elution of a 30 ng DNA sample stored at 56° C. for 72 days. The data show that DNA recovery and the quality of the recovered DNA, as a PCR template, were not affected by raising the storage temperature to 56° C. for 72 days.

### Example 27

### Recovery of Pure DNA from 112 Days and 118 Days of Dry State Storage using a Nanoparticle Matrix Composed of ZrO₂-Passivated with Borax

This experiment is a PCR analysis of DNA recovered from 118 days of dry state storage at 56° C. and 112 days of dry state storage at 56° C. and room temperature. Template DNA samples were adjusted to provide approximately 0.25 ng of DNA per reaction, assuming 100% recovery of the original input DNA. The PCR target sequence was the human amelogenin locus which yields a 558 bp amplicon and is described in detail in Example 16.

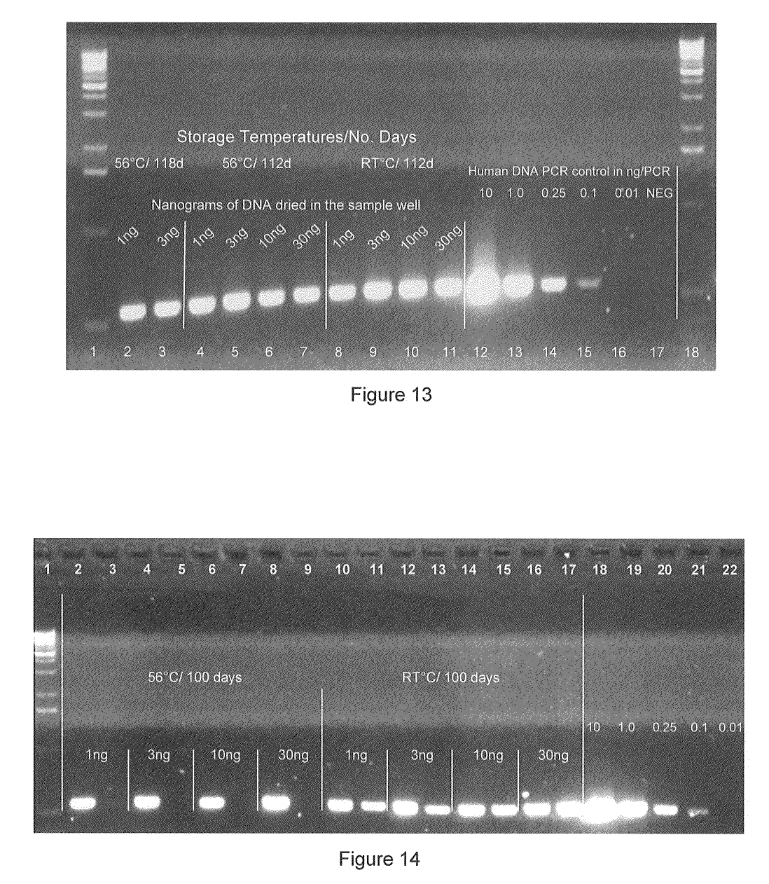

The results are shown in FIG. 13. Lanes 2 and 3 contain DNA samples stored for 118 days at 56° C. Lanes 4-7 contain DNA samples stored for 112 days at 56° C. Lanes 8-11 contain DNA samples stored for 112 days at room temperature. All samples were stored in 0.75× buffer. For the 1 ng samples, a 5 uL, aliquot of the samples was added directly to initiate a 25 μL PCR reaction. For all other samples, a 5 μL aliquot of each sample diluted in 1× Elution Buffer to about 0.05 ng/μL concentration was used to initiate PCR.

The data shows that DNA recovery is quantitative, even for different plates out to 112 days or 118 days stored at 56° C. For accelerated testing of DNA stability, storage at 56° C. is compared to storage at room temperature. Storage for 118 days at 56° C. is predicted to be roughly equivalent to storage for about 20.6 years at room temperature, and storage for 112 days at 56° C. is predicted to be roughly equivalent to storage for about 19.6 years at room temperature.

### Example 28

### Recovery of Pure DNA from 100 Days of Dry State Storage Using a Nanoparticle Matrix Compose of ZrO₂ Passivated with Borax

This experiment is a PCR analysis of pure DNA recovered from 100 days of dry state storage at 56° C. and at room temperature using two different storage buffers and two different glycerol ratios. Template DNA samples were adjusted to provide approximately 0.25 ng of DNA per reaction, assuming 100% recovery of the original input DNA. The PCR target sequence was the human amelogenin locus which yields a 558 bp amplicon and is described in detail in Example 16.

The results are shown in FIG. 14. Lanes 2-9 contain DNA samples stored for 100 days at 56° C. Lanes 10-17 contain DNA samples stored for 100 days at room temperature. Lanes 2, 4, 6, 8, 10, 12, 14, and 16 contain DNA samples that were stored in 0.75× buffer, and lanes 3, 5, 7, 9, 11, 13, 15, and 17 contain DNA samples that were stored in 1.5× buffer. The data shows that the 0.75× storage buffer is superior to the 1.5× storage buffer at 56° C.

### Example 29

### Storage of Cell or Tissue Lysates using a Kaolin Particle Matrix

Blood cells, whole blood, frozen blood, or cheek cell samples collected by either mouth wash rinse or swabs, and are lysed by resuspension in a solution containing 20 mM CAPS, 20 mM NaCO₃, 20 mM EDTA, 2% sodium lauroyl sarcosyl, and 1.8 M guanidinium hydrochloride. The resulting solution of lysed cells or tissue is then diluted 1:1 with water. 20% by volume of Savinase protease solution is then added. Savinase is a bacterial protease from Baccillus species used at 16 U/g. Between 1 mg to 5 mg of borate-passivated kaolin is added to this cell lysate and protease solution. The slurry is then aliquoted into one well of a 96 well microtiter plate at a volume not to exceed about 200 μL. The plate is then allowed to dry at room temperature. Alternatively, the plate is placed at 56° C. until dry.

The resulting dry lysate is recovered from a well by the addition of a volume of water equal to the original fluid volume prior to drying (up to 200 μL). The cell lysate is then processed for DNA isolation by dilution to 500 μL with the addition of 10 mM CAPS, 10 mM NaCO₃, 10 mM EDTA, 1% sodium lauroyl sarcosyl, 0.9M guanidinium hydrochloride, and 50 μL of Savinase. The diluted sample is then incubated at room temperature for 30 minutes with periodic mixing until a homogeneous suspension is formed. The suspension is then transferred to a new vessel, then further incubated for one hour up to 16 hours at 56° C. Kaolin particles are removed from the Savinase digestion product supernatant by centrifugation for 5 minutes at 5,000 G. The resulting DNA-containing supernatant is then transferred to standard microfuge tube. To this supernatant is added up to 1 mg of phosphate-passivated kaolin nanoparticles. LiCl (from a 10 M solution) is added to a final concentration of 0.5 M, followed by the addition of 1 volume of isopropanol. This is mixed to form a suspension. The suspension is incubated for 30 minutes at room temperature, and then centrifuged at 4,000 G for 5 minutes to form a nanoparticle pellet at the bottom of the tube. The DNA-containing nanoparticle pellet is retained. The pellet is then treated with a solution of 50% ethanol/0.15 M NaCl and then centrifuged at 4,000 G for 2 minutes. The pellet is retained and air dried for ten minutes. To the air-dried pellet is added at least 20 μL up to about 200 μL of an elution buffer comprising 10 mM Na Borate at pH 9 and 0.1 mM EDTA. The particles are resuspended in this buffer for 15 to 30 minutes at 56° C. and mixed until a colloidal suspension is reformed. The particles are sedimented by centrifugation at 4,000 G for 5 minutes, and the DNA containing supernatant is harvested by pipetting. DNA in that final eluate is then ready for use for applied genetic analysis or preparative DNA biochemistry.

### Example 30

### Recovery of Crude Buccal Lysate DNA from 10 Days of Dry State Storage using a Kaolin Particle Matrix

DNA extracts of cell lysates from buccal cell wash and buccal swab samples were stored in a kaolin nanoparticle matrix as described in Example 29 for 10 days at room temperature. DNA was purified using Argylla's DNA PrepParticle MicroKit (available at Argylla.com). The PCR target sequence was the human amelogenin locus which yields a 558 bp amplicon and is described in detail in Example 16.

Buccal samples were obtained by buccal cell wash and by buccal swab. For the buccal cell wash, samples were collected from two volunteers, designated A and B. Samples were collected by a 45 second to 1 minute rinse with 10 mL of mouthwash. The buccal cells were pelleted out of solution by centrifugation at 1500 g for 15 minutes. The supernatant was discarded. 1 mL, of DNA Extraction Buffer, containing guanidinium hydrochloride and savinase (16 U/g) at 20% by volume was added to the buccal cell pellet. The cell pellet was resuspended by vortex mixing and incubated at 56° C. overnight (at least 16 hours). Either 125 μL (⅛th) or 35 μL ( 1/30th) of the 1 mL protease digest was dispensed in each well, followed by addition of 20 μL of the nanoparticle suspension. The open plate was then incubated at 56° C. overnight (16 hours) to dry the samples.

For buccal swab samples, buccal cell samples were collected by scraping the inside of the cheek 3 to 4 times using a standard wooden stick with a cotton-head swab. The cotton swab was allowed to dry for approximately four hours at room temperature. The cotton swab was removed from the stick and submerged in 500 μL of 1×DNA extraction buffer containing guanidinium hydrochloride, and savinase (16 U/g) at 20% by volume in a standard 1.5 mL microfuge tube. The samples were incubated at 56° C. overnight (16 hours). The microfuge tubes containing the swabs were spun at 10,000 g for 5 minutes, and the supernatant was transferred to a new tube. The swab was rinsed with an additional 500 μL 1× DNA extraction buffer containing with guanidinium hydrochloride, spun, and the supernatant was combined with the original supernatant. Approximately 250 μL or ¼ of the of the 1 mL supernatant was dispensed in each well, followed by addition of 20 μL of the kaolin nanoparticle suspension. The open plate was incubated at 56° C. for overnight (16 hours) to dry the samples.

For both types of buccal samples (buccal cell wash and buccal swab), to rehydrate the samples, 100 μL of distilled water was added to each well and incubated for 15 minutes at room temperature. The dried sample was resuspended in this volume by repeated pipetting, and the liquified sample was then transferred to a new microfuge tube. The wells were rinsed with another 100 μL of water, and the additional 100 μL was combined with the original sample in the microfuge tube. For the 64 μL and 96 μL, samples, the well was rinsed a second time with an additional 100 μL of water. All rinses and liquified sampled were combined and mixed until a smooth slurry was formed. The volume for all samples was adjusted to a final volume of 500 μL with 1× DNA extraction buffer. The samples were then incubated at 56° C. for 30 minutes.

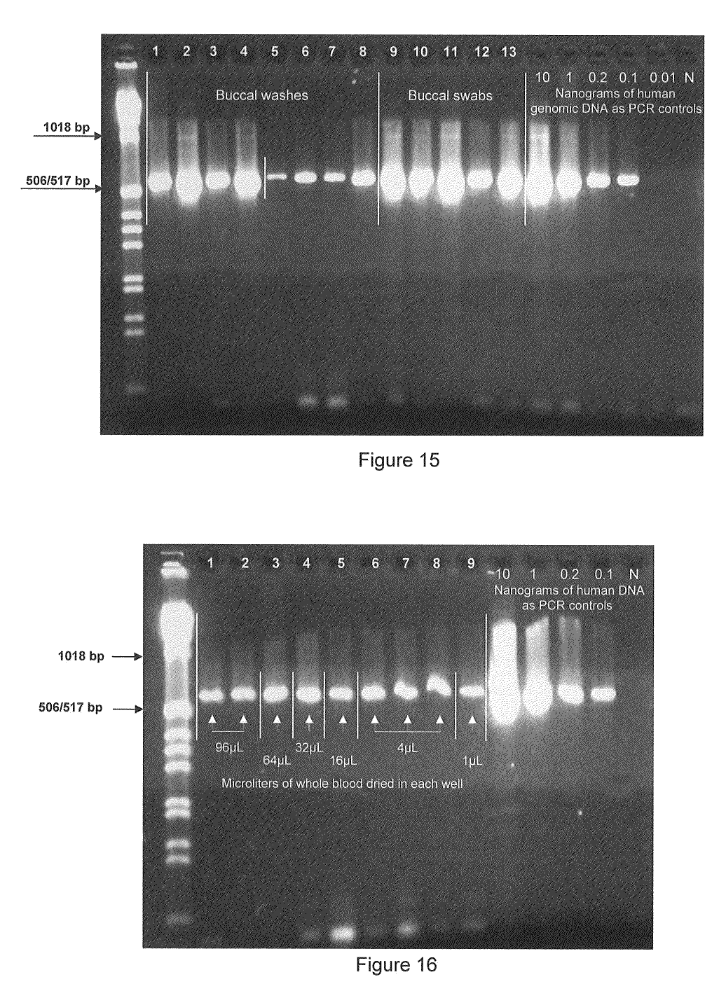

The DNA samples were purified and concentrated for use in the PCR analysis. Each PCR reaction was initiated with approximately 0.2 ng DNA, assuming that one microliter of whole blood contained 30 ng of DNA. The PCR target sequence was the human amelogenin locus which yields a 558 bp amplicon and is described in detail in Example 16. The results are shown in FIG. 15.

Referring to FIG. 15, lane 1 contains 1/500 of ⅛th of the buccal wash sample from volunteer A. Lane 2 contains 1/500 of ⅛th of the buccal wash sample from volunteer B. Lane 3 contains 1/500 of 1/30th of the buccal wash sample from volunteer A. Lane 4 contains 1/500 of 1/30th of the buccal wash sample from volunteer B. Lane 5 contains 1/2000 of ⅛th of the buccal wash sample from volunteer A. Lane 6 contains 1/2000 of ⅛th of the buccal wash sample from volunteer B. Lane 7 contains 1/2500 of 1/30th of the buccal wash sample from volunteer A. Lane 8 contains 1/2500 of 1/30th of the buccal wash sample from volunteer B. Lane 9 contains 1/25 of ¼ of the buccal swab sample from volunteer A. Lane 10 contains 1/50 of ¼ of the buccal swab sample from volunteer A. Lane 11 contains 1/50 of ¼ of the buccal swab sample from volunteer B. Lane 12 contains 1/100 of ¼ of the buccal swab sample from volunteer A. Lane 13 contains 1/100 of ¼ of the buccal swab sample from volunteer B.

### Example 31

### Recovery of Crude Blood Lysate DNA from 1 Day of Dry State Storage Using a Kaolin Particle Matrix

This experiment is a PCR analysis of DNA from crude blood lysates recovered from 1 days of dry state storage in a kaolin nanoparticle matrix as described in Example 29. The results are shown in FIG. 16. For each milliliter of whole blood, the sample was digested with protease in 1× DNA extraction buffer, with guanidinium hydrochloride and savinase (16 U/g) at 20% by volume, in a total volume of 3 mL. The samples were incubated at 56° C. overnight (16 hours). The samples were then dispensed into wells followed by the addition of 2× nanoparticle suspension in the following amounts: 10 μL for whole blood samples from 1 μL to 16 μl, (samples 5-9); 20 μL for whole blood samples of 32 μL (sample 4); 40 μL for whole blood samples of 64 μL (sample 3), and for the two 96 μL, whole blood samples, 60 μL was added to sample 1, and 20 μL was added to sample 2. The open plate was then incubated at 56° C. overnight (16 hours) to dry down the samples.

PCR analysis of carried out on DNA recovered from blood samples ranging from 1 μL to 96 μL stored for 12 hours. Template DNA samples were adjusted to provide approximately 0.2 ng of DNA per reaction, based on the assumption that approximately 34 ng of DNA is present in each microliter of whole blood. The PCR target sequence was the human amelogenin locus which yields a 558 bp amplicon and is described in detail in Example 16.

The results are shown in FIG. 16. Each sample was eluted in 50 μL except the sample in lane 9 which was eluted in 25 μL. Lanes 1 and 2 contain PCR product from the 96 μL whole blood samples. Lane 3 contains PCR product from the 64 μL sample. Lane 4 contains PCR product from the 32 μL sample. Lane 5 contains PCR product from the 16 μL sample. Lanes 6-8 contain PCR product from the 4 μL samples. Lane 9 contains PCR product from the 1 μL sample.

### Example 32

### Recovery of Crude Blood Lysate DNA from 10 Days of Dry State Storage Using a Kaolin Particle Matrix

This experiment is a PCR analysis of DNA from crude blood lysates recovered from 10 days of dry state storage at room temperature in a kaolin nanoparticle matrix as described in Example 29. The samples was predigested with savinase overnight at 56° C., and then dried at 56° C. overnight in a 96 well plate within the kaolin nanoparticle matrix. The calculated amount of DNA added to each PCR assay was based on the assumption that one microliter of whole blood contains approximately 30 ng of DNA. Based on this assumption, 16 μL of whole blood should contain about 480 ng of DNA, and 4 μL of whole blood should contain about 120 ng of DNA. Each sample was eluted from the original well in 50 μL of 1× elution buffer. The PCR target sequence was the human amelogenin locus which yields a 558 bp amplicon and is described in detail in Example 16.

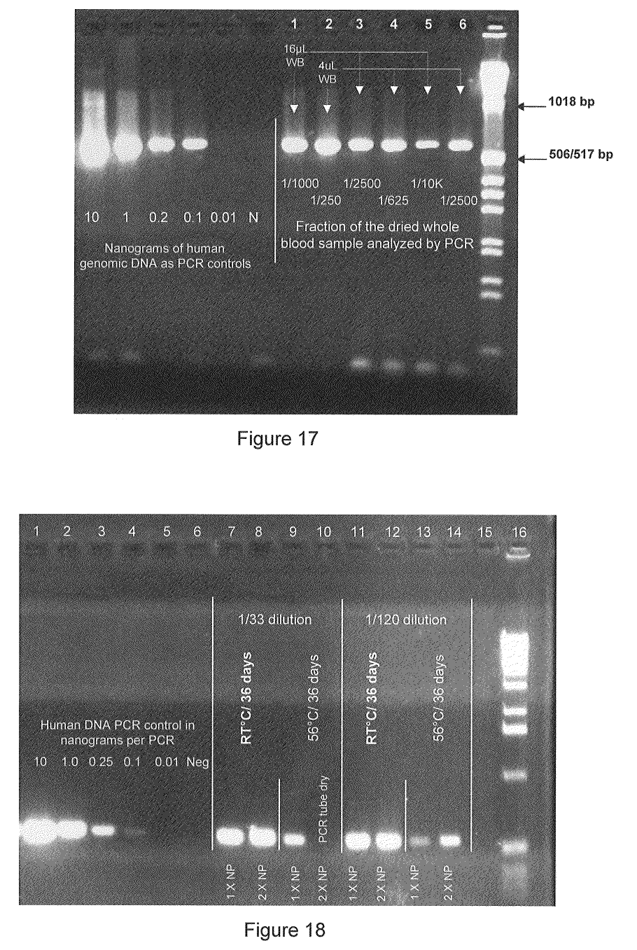

The results are shown in FIG. 17. Lane 1 contains the PCR product from a 1/1000 dilution of a 16 μL sample, corresponding to about 0.48 ng of DNA. Lane 2 contains the PCR product from a 1/250 dilution of a 4 μL sample, corresponding to about 0.48 ng of DNA. Lane 3 contains the PCR product from a 1/2500 dilution of a 16 μL sample, corresponding to about 0.19 ng of DNA. Lane 4 contains the PCR product from a 1/625 dilution of a 4 μL sample, corresponding to about 0.19 ng of DNA. Lane 5 contains the PCR product from a 1/10,000 dilution of a 16 μL sample, corresponding to about 0.048 ng of DNA. Lane 6 contains the PCR product from a 1/2500 dilution of a 4 μL sample, corresponding to about 0.048 ng of DNA. The results show that even extreme small amounts of DNA yield PCR products similar to the purified DNA standards.

### Example 33

### Recovery of Whole Blood DNA from 36 Days of Dry State Storage Using a Kaolin Particle Matrix

This experiment is a PCR analysis of DNA from whole blood recovered from 36 days of dry state storage at room temperature and at 56° C. in a kaolin nanoparticle matrix as described in Example 29. 50 μL of whole blood samples were digested in DNA extraction buffer with the protease, savinase overnight at 56° C. Then the samples were added to the nanoparticle storage matrix that had been previously dried onto plates and mixed with the nanoparticle storage matrix. The samples were then dried at 56° C. overnight. After storage for 36 days, the DNA samples were eluted from the original well in 50 μL of 1× elution buffer. The PCR target sequence was the human amelogenin locus which yields a 558 bp amplicon and is described in detail in Example 16.

The results are shown in FIG. 18. The PCR product from samples stored for 36 days at room temperature are in lanes 7, 8, 11, and 12. The PCR product from samples stored for 36 days at 56° C. are in lanes 9, 13, and 14. The sample for lane 10 was dried up during PCR. Lanes 7, 9, 11, and 13 are from samples stored with a 1× nanoparticle matrix. Lanes 8, 10, 12, and 14 are from samples stored with a 2× nanoparticle matrix. The samples in lanes 7-10 were adjusted to contain about 0.9 ng DNA per PCR assay, based on the assumption that 1 μL of whole blood contains about 30 ng of DNA. The samples in lanes 11-14 were adjusted to contain about 0.25 ng DNA per PCR assay. The data shows that the 2× nanoparticle composition yields more DNA with storage at 56° C., but the two concentrations appear more similar with storage at room temperature.

### Example 34

### Recovery of Fresh Buccal Sample DNA from Storage using a Kaolin Particle Matrix

This experiment is a PCR analysis of DNA from buccal samples recovered from dry state storage using a kaolin nanoparticle matrix as described in Example 29. Eleven cytobrush buccal samples were obtained from volunteer donors. The crude samples were dried for several days and reconstituted in microfuge tubes containing DNA preservation buffer (molecular biology grade water, DNA extraction buffer, sarcosyl solution, and guandinium hydrochloride). Savinase was added to the tubes in order to remove protein contamination. Control aliquots were taken to test the quality of the DNA prior to dry state storage of the DNA. Two methods were used to extract DNA from the crude samples: a modified Qiagen QIAamp DNA Blood Mini Kit protocol and the Argylla DNA PrepParticle MicroKit protocol. The remaining crude sample was combined with the nanoparticle suspension, plated in wells in triplicate, and dried. One well from each sample triplicate remained on the nanoparticle storage plate and was storaged at room temperature for rehydration and testing at a later date. The other two wells for each of the eleven buccal samples were rehydrated, and DNA was extracted using the Qiagen and Argylla kits. The PCR target sequence was the human amelogenin locus which yields a 558 bp amplicon and is described in detail in Example 16.

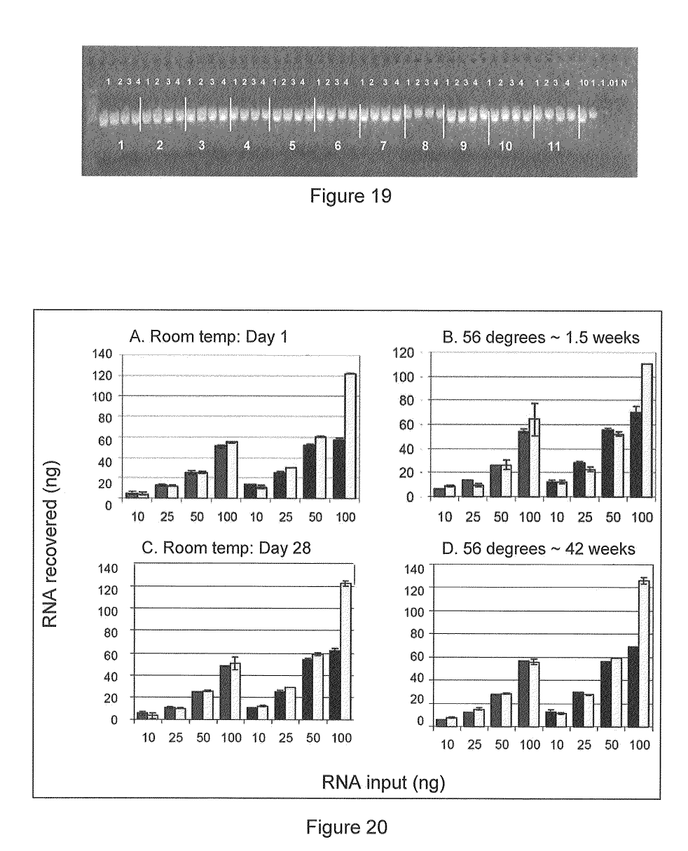

Results are shown in FIG. 19. Each of the 11 samples was treated in 4 different manners. The samples were stored for 4 hours at 56° C. For each of the 11 samples, lane 1 contains DNA that was not plated with the nanoparticle matrix and was eluted in 20 μL using the Argylla DNA PrepParticle MicroKit protocol, with 1 μL, used to initiate the PCR assay. Lane 2 contains DNA that was not plated with the nanoparticle matrix and was eluted in 100 μL using the Qiagen protocol, with 5 μL, was used to initiate the PCR assay. Lane 3 contains DNA that was plated with the nanoparticle matrix, partially dried, hydrated in a 200 μl volume, eluted in 20 μL using the Argylla protocol, with 1 μL was used to initiate the PCR assay. Lane 4 contains DNA that was plated with the nanoparticle matrix, partially dried, hydrated in a 200 μl volume, eluted in 100 μL using the Qiagen protocol, with 5 μL was used to initiate the PCR assay. The expected 558 bp amplicon is present in all four treatments from all 11 donor samples.

This finding indicates that the nanoparticle dry storage method provides a consistent, replicable DNA yield from numerous crude buccal samples. It also illustrates that the DNA yield after nanoparticle dry storage is comparable to that of newly collected samples prior to storage. These results were verified in a separate PCR reaction using a set of GST multiplex primers (data not shown).

### Example 35

### Recovery of RNA from Dry State Storage using a Nanoparticle Matrix Composed of ZrO₂ Passivated with Borax

Two total RNA samples were used for assessing the ability to recover RNA from the nanoparticle storage matrix in sufficient quantity and quality for both RT-PCR and real-time quantitative PCR analysis. The two RNA samples were (1) a purchased fetal liver total RNA with no added RNA stabilizing agents, and (2) a pooled total RNA extract from 50 μL bloodstains obtained from three male newborn (1-day old) individuals solubilized in an RNA stabilizing solution. Two polypropylene, round-bottom 96-well plates containing 2 mm diameter white ceramic disks placed at the bottom of each well were coated with the dried nanoparticle matrix. Then varying amounts of each RNA sample (10 ng, 25 ng, 50 ng, and 100 ng), concentrated in 25 μL nuclease-free water, was added to the nanoparticle matrix. The plates were allowed to dry at room temperature in Bitran bags containing desiccant pouches. The dried plates were sealed with an adhesive cover and stored at either room temperature or at 56° C., to simulate “accelerated” extended storage conditions. Duplicate RNA samples were stored at −20° C. for RNA stability controls. After 1, 3, 7, 14, 21, and 28 days of incubation, the RNA was eluted from the nanoparticle plates by rehydrating the plates with nuclease-free water and then briefly centrifuging the slurry. The supernatant containing the RNA was vacuum centrifuged and resuspended in 10 μl of nuclease-free water.

To determine the stability of the two RNA samples in the nanoparticle storage matrix, the RNA was reverse-transcribed into a cDNA template and two duplex amplification reactions were performed. The first reaction was PCR using a duplex amplification reaction to analyze the stability of a ubiquitously expressed housekeeping gene, GNAS, and a tissue specific gene transcript, the gamma newborn isoform, HBG2n3 f. The second reaction was quantitative real-time PCR (qPCR) using a published duplex reaction employed to analyze the stability of a different ubiquitously expressed housekeeping gene, S 15, and an additional tissue specific gamma newborn isoform, HBG1n1g. The PCR and qPCR products were then subjected to gel-based and cycle threshold analysis, respectively, in order to quantify the RNA recovered from each sample.

The plate stored at 56° C. was used to make stability predictions because chemical reaction rates (for first order reactions) generally double with each 10° C. increase. Stability predictions can be made using the Arrhenius Equation: Predicted Stability=Accelerated Stability×2^{DT/10}, where DT=difference between room temperature (22° C.) and sample storage temperature (56° C.).

The results are shown in FIG. 20. The quantity of RNA recovered from the nanoparticle storage matrix and that of the stability control RNA stored at −20° C. was consistent at all time points (t=1, 3, 7, 14, 21, and 28 days) and from both storage conditions (room temperature and 56° C.) tested. Room temperature stability data from the shortest (FIG. 20A) and longest (FIG. 20C) time points tested (t=1 and t=28) is comparable to that at 56° C. for the same time points (FIGS. 20B and 20D). The consistency of these RT-PCR results illustrates the long-term stability of RNA stored in the nanoparticle storage matrix.

In FIG. 20, the first four paired columns illustrate the RNA yield from fetal liver total RNA samples in the absence of an RNA-stabilizing agent, while the second four paired columns illustrate the RNA yield from newborn (1-day-old) whole blood RNA in the presence of an RNA-stabilizing agent. In both cases, the amount of RNA recovered from the nanoparticle storage matrix is very similar. Therefore, the presence or absence of an RNA-stabilizing agent does not seem to have a significant effect on the amount of RNA eluted from the nanoparticle storage matrix.

Stored RNA (>25 ng) was readily detectable and amplifiable after 28 days of ambient and 56° C. storage with both PCR (FIGS. 20C and 20D) and quantitative real-time PCR (not shown) reactions. The 28 day 56° C. incubation was equivalent to approximately 42 weeks of room temperature storage.

Both the RT-PCR and real-time qPCR amplification reactions yielded two distinct bands after gel electrophoresis and cycle threshold values, respectively, in all tested samples, at all time points, with both storage conditions (not shown). This result suggests that the RNA eluted from the nanoparticle storage matrix is both of high quality and sufficient quantity for sensitive amplification assays, even after storage for extended periods.

### Example 36

### DNA Storage on Particle Matrix Plates

DNA is stored by adding to 100 μL or less of purified DNA (up to 1 μg), 125 μg of borate-passivated zirconium, 40 μg of disodium tetraborate (borax), and 75 μg of glycerol as a 20 μL suspension. After adding the mixture to the DNA, allow to air dry, and store at room temperature. To recover the DNA, add 10 μL to 100 μL of water and resuspend the particle to suspension. Pellet the nanoparticle from the suspensions and remove the DNA solution.

### Example 37

### RNA Storage on Particle Matrix Plates

RNA Storage on nanoparticle plates is performed as described in Example 36, except with RNA rather that with DNA. RNA is stored by adding to 100 μL or less of purified RNA (up to 1 μg), 125 μg of borate-passivated zirconium, 40 μg of disodium tetraborate (borax), and 75 μg of glycerol as a 20 μL suspension. After adding the mixture to the RNA, allow to air dry, and store at room temperature. To recover the RNA, add 10 μL to 100 μL of water and resuspend the particle to suspension. Pellet the nanoparticle from the suspensions and remove the RNA solution.

### Example 38

### Storage of Total Serum Proteins on Particle Matrix Plates

For 50 μL of fluid serum (5 mg of total protein), 50 μL of a suspension consisting of 1 mg of borate-passivated kaolin (Example 2), 2 mg of borax, and 3.5 mg of glycerol is added, and the mixture is allowed to air dry. To recover the serum proteins, water is added to at least 20 μL, and the particles are gently agitated back into colloidal suspension.

### Example 39

### Storage of Serum Antibody on Particle Matrix Plates

For 100 μL of serum, add 50 μL of 2 M sodium sulfate, up to 20 mg of kaolin nanoparticles (200 nm diameter) coated with mercaptosilane, further functionalized in sequential order with divinyl sulfone, followed by mercaptoethanol. After the serum proteins adsorb to the surface ligands, these ligand bound proteins are concentrated and enriched, first by sedimentation of particles from suspension. Then these particles are washed in 100 mM NaCl three times by sedimentation-resuspension and are added to the 100 μL of serum diluted in PBS to 800 mM in which 200 μL of 2 M sodium sulfate solution is added. The thiophilic ligand coated kaolin particles are added as a suspension in PBS with 0.5 M sodium sulfate in a volume not to exceed 500 μL. After at least a 30 minute incubation, the particles are sedimented at 4000 G for 5 minutes, resuspended in PBS and 0.5 M sodium sulfate and sedimented at 4000 G. The resulting pellet is resuspended in 100 μl, of a solution containing 2 mg of thiophilic kaolin, 2 mg of borax, and 3.5 mg of glycerol. The suspension is allowed to dry at room temperature to dryness. The immunoglobulins are recovered by resuspending the pellet in at least 50 μL, of 100 mM NaCl.

### Example 40

### Dry State Storage of Cell or Tissue Lysates with a Non-Ceramic Particle Matrix

The process is as described in Example 29, except that the particle matrix comprises particles composed of polysucrose, such as the sucrose-epichlorohydrin polymer, Ficoll.

### Example 41

### Dry State Storage of Cell or Tissue Lysates using Non-Ceramic Particles of Dimension Greater than 1 Micron

The process is the same as that described in Example 29, except that the particle matrix comprises of spherical particles composed of an epoxide cross linked polymer of agarose, such as the beads under the commercial name of Sephadex-50.

### Example 42

### Kaolin Particle Matrix Kits for Storage of Cell or Tissue Lysates

The kaolin particle matrix kits are particularly useful for storage of DNA from crude samples such as blood cells, whole blood, frozen blood, cheek cell samples, and other tissue samples. As configured in this Example, a single kit, as shown in Table 1 is useful for one hundred (50) 100 μL samples (5 ml). The individual kit components are described further in Example 29. Generally, an additional 10% of each kit component is provided.

TABLE 1

| | | | |
|---|---|---|---|
| | Volume | Volume | |
| Component | Needed | Provided | Container |

| | | | | | | |
|---|---|---|---|---|---|---|
| 20x Sarkosyl | 500 | μL | 550 | μL | 1.5 | mL tube |
| 20x Extraction | 500 | μL | 550 | μL | 1.5 | mL tube |
| Buffer | | | | | | |
| 7.5M Guanidinium | 1.2 | mL | 1.32 | mL | 1.5 | mL tube |
| HCl | | | | | | |

| | | | | | |
|---|---|---|---|---|---|
| Kaolin 50 mg/mL | 2.0 | mL | 2.2 | mL | two (2) |
| | | | | | 1.5 mL tubes |

The components used to make 6 kits are shown in Table 2.

TABLE 2

| | Volume | | Volume | | | | Total for | |
|---|---|---|---|---|---|---|---|---|
| Component | Needed | | Provided | | Container | | 6 Kits | |
| 20x Sarkosyl | 500 | μL | 550 | μL | 1.5 | mL tube | 3.3 | ml |
| 20x Extraction | 500 | μL | 550 | μL | 1.5 | mL tube | 3.3 | ml |
| Buffer | | | | | | | | |
| 7.5M Guanidinium | 1.2 | mL | 1.32 | mL | 1.5 | mL tube | 7.29 | ml |
| HCl | | | | | | | | |

| | | | | | | | |
|---|---|---|---|---|---|---|---|
| Kaolin 50 mg/mL | 2.0 | mL | 2.2 | mL | two (2) | 13.2 | ml |
| | | | | | 1.5 mL tubes | | |

All references cited in this application are incorporated by reference herein in their entireties. While the present invention has been described with reference to its preferred embodiments and the foregoing non-limiting examples, those skilled in the art will understand and appreciate that the scope of the present invention is intended to be limited only the claims appended hereto.

1. A chemical formulation for dry state storage of biomolecules comprising:
a. a plurality of surface non-porous nanoparticles, wherein the non-porous nanoparticles have a longest dimension of less than about 1 micron (μm);
b. at least one small molecule filler, wherein the at least one small molecule filler has approximately the same applied mass as the nanoparticles, and wherein upon drying, the at least one small molecule filler occupies the unoccupied void volume between closely packed nanoparticles in a dried state, thereby forming a nanoparticle complex; and
the resulting nanoparticle complex forming a dense, closely packed matrix in which biomolecules can be sequestered in the space between the nanoparticles.

2. The formulation of claim 1, wherein the non-porous nanoparticles are selected from the group consisting of aluminosilicates and metal oxides.

3. The formulation of claim 2, wherein the non-porous nanoparticles comprise of metal oxides selected from the group consisting of aluminum oxide, titanium oxide, tungsten oxide, zirconium oxide, tin oxide, and combinations thereof.

4. The formulation of claim 2, wherein the non-porous nanoparticles comprise of aluminosilicates selected from the group consisting of phyllosilicates, smectites, and combinations thereof.

5. The formulation of claim 4, wherein the aluminosilicates comprise of clays, and wherein the clays are further selected from the group consisting of kaolin and bentonite.

6. The formulation of claim 1, where the nanoparticles are passivated with borate.

7. The formulation of claim 1, wherein the at least one small molecule filler is selected from the group consisting of sodium borate, boric acid-glycerol, boric acid-1,3 propane-diol, sodium phosphate, and combinations thereof.

8. The formulation of claim 1, wherein the nanoparticle complex forms a dried film or pellet applied to the bottom of a microtiter plate.

9. The formulation of claim 1, wherein the nanoparticle complex forms a dried film or pellet applied to the bottom of a separable storage tube.

10. The formulation of claim 1, wherein the biomolecules are DNA.

11. The formulation of claim 1, wherein the biomolecules are RNA.

12. The formulation of claim 1, wherein the biomolecules are proteins.

13. The formulation of claim 12, wherein the proteins are immunoglobulins.

14. The formulation of claim 1, wherein the at least one small molecule filler is selected from the group consisting of sodium borate, CAPS, NaCO₃, EDTA, sodium lauroyl sarcosyl, guanidinium hydrochloride, and combinations thereof.

15. The formulation of claim 14, wherein the biomolecules further comprise is cell lysates.

16. The formulation of claim 14, wherein the biomolecules are selected from the group consisting of blood, blood components, buccal cells, cells from in vitro culture, and solid tissue lysates and homogenates, and wherein the blood components are further selected from the group consisting of serum, plasma, and lymphocytes.

17. A chemical formulation for dry state storage of biomolecules comprising:
a. a plurality of surface porous particles, wherein the particles have a longest dimension of less than about 1 micron, and wherein the particles disperse into discrete particles upon hydration;
b. at least one small molecule filler, wherein the at least one small molecule filler has approximately the same applied mass as the particles, and wherein upon drying, the at least one small molecule filler occupies the unoccupied void volume between closely packed nanoparticles in a dried state, thereby forming a particle complex; and
the resulting particle complex forming a dense, closely packed matrix in which biomolecules can be sequestered in the space between the particles.

18. A chemical formulation for dry state storage of biomolecules comprising:
a. a plurality of surface porous particles, wherein the particles have a longest dimension of between about 1 micron and about 50 microns, and wherein the particles disperse into discrete particles upon hydration;
b. at least one small molecule filler, wherein the at least one small molecule filler has approximately the same applied mass as the particles, and wherein upon drying, the at least one small molecule filler occupies the unoccupied void volume between closely packed nanoparticles in a dried state, thereby forming a particle complex; and
the resulting particle complex forming a dense, closely packed matrix in which biomolecules can be sequestered in the space between the particles.


SEQUENCE LISTING (2 sequences)

SEQ ID NO: 1 - DNA, 27 bp, Homo sapiens
agatgaagaatgtgtgtgatggatgta

SEQ ID NO: 2 - DNA, 24 bp, Unknown
  a nuclear chromosome encoded gene, amylogenin, encoded on both the X and Y chromosome
gggctcgtaaccataggaagggta